(19) United States
(12) Patent Application Publication
(10) Pub. No.: US 2023/0225623 A1
(43) Pub. Date: Jul. 20, 2023

(54) Methods And Systems For Non-Invasive Cuff-Less Blood Pressure Monitoring

(71) Applicants: Northwestern University, Evanston, IL (US); Georgia Tech Research Corporation, Atlanta, GA (US)

(72) Inventors: Andrew M. Carek, Evanston, IL (US); James A. Heller, Evanston, IL (US); Mozziyar Etemadi, Evanston, IL (US); Avidor B. Kazen, Evanston, IL (US); Omer T. Inan, Atlanta, GA (US); Venu G. Ganti, Atlanta, GA (US)

(21) Appl. No.: 17/906,672

(22) PCT Filed: Mar. 19, 2021
(86) PCT No.: PCT/US2021/023126

§ 371 Date: Sep. 19, 2022

Related U.S. Application Data
(60) Provisional application No. 62/992,338, filed on Mar. 20, 2020.
(60) Provisional application No. 62/992,196, filed on Mar. 20, 2020.

(51) Int. Cl. A61B 5/021; A61B 5/00

(52) U.S. Cl. CPC A61B 5/02108; A61B 5/6802; A61B 5/318

(57) ABSTRACT

An exemplary embodiment of the present disclosure provides systems and methods for non-invasively measuring blood pressure, the system and methods comprise a wearable device having a first surface, a first sensor positioned on the first surface of the wearable device, the first sensor configured to receive a first signal, wherein the first signal is indicative of a first blood-volume change in a first vessel of a subject, a second sensor positioned within the wearable device, the second sensor configured to receive a second signal, wherein the second signal is indicative of a cardiac mechanical motion of the subject, and a processor positioned within the wearable device, the processor configured to generate an output based at least on the first signal and the second signal, the output representing a blood pressure measurement of the subject.

### CROSS-REFERENCE TO RELATED APPLICATIONS

This application claims the benefit of U.S. Provisional Application Serial Nos. 62/992,338, filed on 20 Mar. 2020, and 62/992,196, filed on 20 Mar. 2020, which are incorporated herein by reference in their entirety as if fully set forth below.

### FEDERALLY SPONSORED RESEARCH STATEMENT

This invention was made with government support under grant/award number 1U01EB018818-01 awarded by the National Institutes of Health, grant/award number 5R01HL130619-03, awarded by the National Institutes of Health, and grant/award number 1U54EB027690, awarded by the National Institutes of Health. The government has certain rights in the invention.

### FIELD OF THE DISCLOSURE

The various embodiments of the present disclosure relate generally to methods, systems, and devices for measuring hemodynamic variables to extract a blood pressure measurement in a subject, and more particularly to extracting blood pressure measurements from pulse transit time (PTT).

### BACKGROUND

Blood pressure abnormalities, such as hypotension and hypertension, are major health care concerns afflicting nearly half of the US population. To exacerbate matters, hypertension is the leading risk factors for cardiovascular diseases and can lead to end organ damage such as heart failure, kidney failure, vascular aneurysm, and retinopathy, among others. To diagnose and manage hypertension, clinicians rely on both clinic and home readings of blood pressure (BP) to property develop management plans. For most people, BP measurements are typically limited to semi-regular clinical visits, but even BP measurements several times a year does not provide insight to dynamic and varying actual blood pressure that can change drastically even within minutes. Therefore, there is a need to provide a more complete picture of heart health and hemodynamics through portable and at-home measurements.

The gold-standard for monitoring BP is through an invasive intra-arterial catheter with an attached strain gauge, such as a radial artery pressure measurement commonly used in hospital settings. Other techniques involve an automatic cuff-based oscillometry device that can attach to a limb (e.g., upper arm). This method requires the subject to remain still as the cuff self-inflates and slowly deflates to measure the arterial pulsation through the blood vessels of the limb. While the oscillometry device can be used at home, it is bulky and requires a lengthy procedure where the subject must remain in a static position and can sometimes be painful as the cuff applies pressure above the occlusion level of the blood vessel. A cuff-based wristwatch can reduce the bulkiness of a device, but the measurement of BP in the wrist blood vessels have shown significantly lower accuracies compared to upper-arm BP cuffs (see M. Kikuya, K. Chonan, Y. Imai, E. Goto, M. Ishii, et al., “Accuracy and reliability of wrist-cuff devices for self-measurement of blood pressure,” Journal of Hypertension, vol. 20, no. 4, pp. 629-638, 2002.). Importantly, devices based on oscillometric cuffs rely solely on the pulse amplitude, tend to be inaccurate in subjects with weak pulses from disorders such as atherosclerosis or obesity, and fail to provide continuous BP measurements.

Cuff-less home monitoring solutions for estimating BP could provide continuous, or frequent, measurements of blood pressure and allow for improved detection and management of blood pressure abnormalities. Blood pressure can be estimated based on hemodynamic variables of pulse arrival time (PAT), pre-ejection period (PEP), pulse wave velocity (PWV), and pulse transit time (PTT) using on-body sensors, such as a photoplethysmograph (PPG) in combination with other sensors. Several BP monitoring systems have been proposed based on one or more of these hemodynamic variables; however, these systems require a combination of sensors on a combination of two or more devices, such as oscillometric cuffs, mobile phone sensors, weight scales, beds or platforms with sensors, wearable patches, wearable glasses, finger pulsometers, and/or wearable watches. To enhance the reliability of a subject to consistently measure their BP, there is a need to simplify the device used to estimate the subject’s BP. Described herein, the combination of sensors in a single wearable device can reliably detection various hemodynamic variables, including PAT, PEP, and/or PTT to assist in the diagnosis and management of hypertension of a subject.

### BRIEF SUMMARY

The present disclosure relates to a system for non-invasively measuring blood pressure. An exemplary embodiment of the present disclosure provides a system comprising a wearable device having a first surface, a first sensor, a second sensor, and a processor. The first sensor can be positioned on the first surface of the wearable device. The first sensor can be configured to receive a first signal. The first signal can be indicative of a first blood-volume change in a first vessel of a subject. The second sensor can be positioned within the wearable device. The second sensor can be configured to receive a second signal. The second signal can be indicative of a cardiac mechanical motion of the subject. The processor can be positioned within the wearable device. The processor can be configured to generate an output based at least on the first signal and the second signal. The output can represent a blood pressure measurement of the subject.

In any of the embodiments disclosed herein, the system can further comprise an actuator configured to emit a measuring signal. The received first signal can be based at least in part on the emitted measuring signal.

In any of the embodiments disclosed herein, the first vessel can comprise a peripheral vessel of an ear, a nasal septum, a forehead, a sternum, a fingertip, a wrist, a toe, a foot, or any combination thereof, of the subject.

In any of the embodiments disclosed herein, the first sensor can comprise a photodetector. The first signal can comprise light.

In any of the embodiments disclosed herein, the actuator can comprise a light source. The measuring signal can comprise light.

In any of the embodiments disclosed herein, the first sensor can comprise at least one of a light source and/or a photodetector.

In any of the embodiments disclosed herein, the first sensor can further be configured to receive one or more wavelengths of the first signal.

In any of the embodiments disclosed herein, the one or more wavelengths of the first signal can range from about 1000 nm to about 200 nm.

In any of the embodiments disclosed herein, the first sensor can be configured to receive a first photoplethysmograph (PPG) signal of the first vessel.

In any of the embodiments disclosed herein, the system can further comprise a third sensor positioned on a second surface of the wearable device.

In any of the embodiments disclosed herein, the third sensor can comprise at least one of a light source and/or a photodetector.

In any of the embodiments disclosed herein, the third sensor can be configured to emit and/or receive a third signal. The third signal can be indicative of a second blood-volume change in a second vessel of the subject.

In any of the embodiments disclosed herein, the system can further comprise an actuator configured to emit a measuring signal. The received third signal can be based at least in part on the emitted measuring signal.

In any of the embodiments disclosed herein, the third sensor can comprise a photodetector. The actuator can comprise a light source. The third signal and the measuring signal can each comprise light.

In any of the embodiments disclosed herein, the third signal and the measuring signal can each comprises one or more wavelengths of light.

In any of the embodiments disclosed herein, the second vessel can comprise a peripheral vessel of an ear, a nasal septum, a forehead, a sternum, a fingertip, a wrist, a toe, a foot, or any combination thereof, of the subject.

In any of the embodiments disclosed herein, the third sensor can be configured to receive a second PPG signal of the second vessel.

In any of the embodiments disclosed herein, -the first vessel and the second vessel can comprise different peripheral vessels of the subject.

In any of the embodiments disclosed herein, the third sensor can be further configured to emit and/or receive one or more wavelengths of the third signal.

In any of the embodiments disclosed herein, each of the one or more wavelengths of the third signal can range from about 1000 nm to about 200 nm.

In any of the embodiments disclosed herein, the second sensor can comprise an accelerometer, a magnetometer, a digital camera, a microphone, an optical sensor, or combinations thereof.

In any of the embodiments disclosed herein, the second sensor can be configured to receive a seismocardiograph (SCG) signal of the subject.

In any of the embodiments disclosed herein, the system can further comprise a fourth sensor positioned on the second surface of the wearable device. The fourth sensor can be configured to receive a fourth signal. The fourth signal can be indicative of a first electrical activity of the subject.

In any of the embodiments disclosed herein, the fourth sensor can be configured to receive an electrocardiogram (ECG) signal of the subject.

In any of the embodiments disclosed herein, the fourth sensor can be configured to receive an impedance cardiogram (ICG) signal of the subject.

In any of the embodiments disclosed herein, the fourth sensor can be configured to receive an impedance plethysmogram (IPG) signal of the subject.

In any of the embodiments disclosed herein, the system can comprise a fifth sensor positioned on the second surface of the wearable device. The fifth sensor can be configured to receive a fifth signal. The fifth signal can be indicative of a second electrical activity of the subject.

In any of the embodiments disclosed herein, the fifth sensor can be configured to receive one of an ECG signal, an ICG signal, or an IPG signal of the subject.

In any of the embodiments disclosed herein, the system can further comprise a sixth sensor positioned within the wearable device. The sixth sensor can be configured to receive a sixth signal. The sixth signal can be indicative of a mechanical motion of the subject.

In any of the embodiments disclosed herein, the sixth signal can be configured to receive a gyrocardiogram (GCG) signal of the subject.

In any of the embodiments disclosed herein, the system can further comprise correlating the first signal and the second signal to one or more hemodynamic variables.

In any of the embodiments disclosed herein, the one or more hemodynamic variables can comprise a pulse transit time (PTT), pulse arrival time (PAT), pre-ejection period (PEP), blood pressure (BP), or a pulse wave velocity (PWV).

In any of the embodiments disclosed herein, the system can further comprise extracting a blood pressure reading from the one or more hemodynamic variables.

In any of the embodiments disclosed herein, the first surface of the wearable device can be configured to be placed in indirect contact with the first vessel of the subject.

In any of the embodiments disclosed herein, the first surface of the wearable device can be configured to be placed in direct contact with a sternum of the subject.

In any of the embodiments disclosed herein, the second surface of the wearable device can be configured to be placed in indirect contact with the second vessel of the subject.

In any of the embodiments disclosed herein, the second surface of the wearable device can be configured to be placed in direct contact with the ear, the nasal septum, the forehead, the fingertip, the wrist, the toe, the foot, or any combination thereof, of the subject.

In any of the embodiments disclosed herein, the wearable device can comprise a wristwatch.

In any of the embodiments disclosed herein, the first surface of the wristwatch can be configured to receive the first and second signals when placed in contact with the sternum of the subject. The second surface of the wristwatch can be configured to receive at least one of the third, fourth, fifth, and/or sixth signals when placed in contact with the ear, the nasal septum, the forehead, the fingertip, the wrist, the toe, the foot, or any combination thereof, of the subject.

An exemplary embodiment of the present disclosure provides a method for non-invasively measuring blood pressure. The method can comprise receiving a first signal, receiving a second signal, determining a blood pressure measurement of a subject, and outputting the blood pressure measurement of the subject. The first signal can be received by a wearable device. The first signal can be indicative of a first blood-volume change in a first vessel of a subject. The second signal can be received by a wearable device. The second signal can be indicative of a first cardiac mechanical motion of the subject. Determining the blood pressure measurement of the subject can be based on the first and second signals.

In any of the embodiments disclosed herein, the method can further comprise receiving, by the wearable device, a third signal. The third signal can be indicative of a second blood-volume change in a second vessel of the subject.

In any of the embodiments disclosed herein, the method can further comprise receiving the first signal by an accelerometer, a magnetometer, a digital camera, a microphone, an optical sensor, or combinations thereof.

In any of the embodiments disclosed herein, the method can further comprise receiving, by the wearable device, a fourth signal. The fourth signal can be indicative of a first electrical activity of the subject.

In any of the embodiments disclosed herein, the method can further comprise receiving, by the wearable device, a fifth signal. The fifth signal can be indicative of a second electrical activity of the subject.

In any of the embodiments disclosed herein, the method can further comprise receiving, by a wearable device, a sixth signal. The sixth signal can be indicative of a mechanical motion of the subject.

In any of the embodiments disclosed herein, the processor can further be configured to correlate the first signal and the second signal to one or more hemodynamic variables.

In any of the embodiments disclosed herein, the processor can be further configured to extract a blood pressure reading from the one or more hemodynamic variables.

In any of the embodiments disclosed herein, the wearable device can comprise a first surface and a second surface.

In any of the embodiments disclosed herein, the first surface of the wearable device can be configured to receive the first signal and the second signal of the subject.

In any of the embodiments disclosed herein, the method can further comprise indirectly contacting the first surface of the wearable device on the first vessel of the subject.

In any of the embodiments disclosed herein, the method can further comprise directly contacting the first surface of the wearable device on a sternum of the subject.

In any of the embodiments disclosed herein, the second surface of the wearable device can be configured to receive at least one of the third, fourth, fifth, and/or sixth signals of the subject.

In any of the embodiments disclosed herein, the method can further comprise indirectly contacting the second surface of the wearable device on the second vessel of the subject.

In any of the embodiments disclosed herein, the method can further comprise directly contacting the second surface of the wearable device with the ear, the nasal septum, the forehead, the fingertip, the wrist, the toe, the foot, or any combination thereof, of the subject.

In any of the embodiments disclosed herein, the wearable device can comprise a wristwatch having a first face and a second face.

In any of the embodiments disclosed herein, the method can further comprise receiving the first signal by a first sensor positioned on the first face of the wristwatch, receiving a second signal by a second sensor positioned within the wristwatch, and receiving a third signal by a third sensor positioned on the second surface of the wristwatch. The first and second signals can be received when the first face of the wristwatch is positioned to be in contact with a sternum of the subject. The third signal can be received when the third sensor is positioned against the skin of the subject.

An exemplary embodiment of the present disclosure provides a system for non-invasively measuring blood pressure. The system can comprise a wearable device, a first sensor, a second sensor, a third sensor, and a processor. The wearable device can have a first surface and a second surface. The first sensor can be positioned on the first surface of the wearable device. The first sensor can be configured to receive a first photoplethysmograph (PPG) signal. The first PPG signal can be indicative of a first blood-volume change in a first vessel of a subject. The second sensor can be positioned within the wearable device. The second sensor can be configured to receive a seismocardiograph (SCG) signal. The SCG signal can be indicative of a cardiac mechanical motion of the subject. The third sensor can be positioned within and/or on the second surface of the wearable device. The third sensor can be configured to receive one or more of an electrocardiogram (ECG) signal, an impedance cardiogram (ICG) signal, an impedance plethysmogram (IPG) signal, or a gyrocardiogram (GCG) signal. The processor can be positioned within the wearable device. The processor can be configured to determine a blood pressure measurement of the subject based on at least the first PPG signal, the SCG signal, and one or more of the ECG, ICG, IPG, or GCG signals and generate an output representing the blood pressure measurement of the subject.

In any of the embodiments disclosed herein, the system can further comprise a fourth sensor positioned on the second surface of the wearable device. The fourth sensor can be configured to receive a second PPG signal. The second PPG signal can be indicative of a second blood-volume change in a second vessel of the subject.

In any of the embodiments disclosed herein, the second PPG signal can be indicative of a blood-volume change in a vessel of the subject different than the first PPG signal.

In any of the embodiments disclosed herein, the processor can further be configured to transition the wearable device from a normal mode of operation to one or more measurement modes of operation.

In any of the embodiments disclosed herein, the normal mode of operation can comprise detection of the first PPG, the SCG, and the ECG.

In any of the embodiments disclosed herein, the one or more measurement modes of operation can comprise a continuous mode, a pulse transit time (PTT) mode, pulse arrival time (PAT) mode, pre-ejection period (PEP) mode, a blood pressure (BP) mode, and a pulse wave velocity (PWV) mode.

In any of the embodiments disclosed herein, the transition from the normal mode of operation to the continuous mode can comprise initiating the first sensor, the second sensor, and the third sensor of the wearable device.

In any of the embodiments disclosed herein, the processor can be configured to receive the first PPG signal, the SCG signal, and the ECG signal while in continuous mode.

In any of the embodiments disclosed herein, the transition from the normal mode of operation to the PTT mode can comprise the first sensor, the second sensor, and optionally the fourth sensor of the wearable device.

In any of the embodiments disclosed herein, the processor can be configured to receive the first PPG signal, the SCG signal, and optionally the second PPG signal while in PTT mode.

In any of the embodiments disclosed herein, the transition from the normal mode of operation to the PAT mode can comprise the first sensor, the third sensor, and optionally the fourth sensor of the wearable device.

In any of the embodiments disclosed herein, the processor can be configured to receive the first PPG signal, the ECG signal, and optionally the second PPG signal while in PAT mode.

In any of the embodiments disclosed herein, the transition from the normal mode of operation to the PEP mode can comprise the second sensor and the third sensor of the wearable device.

In any of the embodiments disclosed herein, the processor can be configured to receive the SCG signal and the ECG signal while in PEP mode.

In any of the embodiments disclosed herein, the system can further comprise extracting a blood pressure reading from the one or more measurement modes of operation.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 depicts a block diagram of illustrative computing device architecture, according to an example implementation.

FIGS. 2A and 2B show photographs of a wearable device, in accordance with an exemplary embodiment of the present invention.

FIG. 2C provides an exploded view of a wearable device, in accordance with an exemplary embodiment of the present invention.

FIGS. 3A and 3B show the front (FIG. 3A) and back (FIG. 3B) of a sternum PPG board of processor 230, in accordance with an exemplary embodiment of the present invention.

FIGS. 4A and 4B show the front (FIG. 4A) and back (FIG. 4B) of a wrist PPG/ECG board of processor 230, in accordance with an exemplary embodiment of the present invention.

FIG. 5 shows a main board of processor 230, in accordance with an exemplary embodiment of the present invention.

FIGS. 6A and 6B provide an example placement of wearable device 200 for one or more measurements, in accordance with an exemplary embodiment of the present invention.

FIG. 7A provides an example placement of wearable device 200 for one or more measurements, in accordance with an exemplary embodiment of the present invention.

FIG. 7B provides an example placement of sensors on wearable device 200, in accordance with an exemplary embodiment of the present invention.

FIG. 7C shows example SCG signal and PPG signal for extracting a PTT measurement, in accordance with an exemplary embodiment of the present invention.

FIG. 8 provides a block diagram of signal processing technique to extract the AO peak using both the SCG signal and the PCG signal, in accordance with an exemplary embodiment of the present invention.

FIG. 9 provides waveforms for an ECG signal, an SCG signal, a GCG signal, a sternum PPG signal, and a wrist PPG signal, in accordance with an exemplary embodiment of the present invention.

FIG. 10A shows waveforms for an SCG signal and a sternum PPG signal to extract a blood pressure measurement, in accordance with an exemplary embodiment of the present invention.

FIG. 10B shows waveforms for an ECG signal, an SCG signal, a GCG signal, a sternum PPG signal, and a wrist PPG signal to extract a blood pressure measurement, in accordance with an exemplary embodiment of the present invention.

FIG. 11 provides a block diagram of illustrative wearable device mode transition method 1100, according to an example implementation.

FIG. 12 provides an example measurement placement and protocol, according to an example implementation.

FIGS. 13A and 13B show plots of subject versus MAP Error (mmHg), DP Error (mmHg), and SP Error (mmHg) for PTT measurement and PAT measurement, representing a comparison between PTT and PAT during rest (FIG. 13A) and cold pressor (FIG. 13B), in accordance with an exemplary embodiment of the present invention.

FIG. 14 shows a box-plot showing the statistically significant (*p<0.05) decreasing root-mean-square-error (RMSE) in intrasubject testing loss with an increasing number of calibration points, in accordance with an exemplary embodiment of the present invention.

FIG. 15 shows a box-plot showing the notable differences in root-mean-square-error (RMSE) in intrasubject testing loss between the regular intrasubject calibration method, the global y-intercept model, and the global slope model with an increasing number of calibration points, in accordance with an exemplary embodiment of the present invention.

FIG. 16 shows a box-plots showing statistical significance (*p<0.05) in root-meansquare-error (RMSE) intrasubject testing loss between two different two-point calibration methods using either the maximum and minimum blood pressure (BP) values or pulse transit time (PTT) values and the standard multi-point calibration method, in accordance with an exemplary embodiment of the present invention.

FIG. 17A shows correlation and Bland-Altman plots for mean arterial pressure (MAP), diastolic pressure (DP), and systolic pressure (SP) for a study having 13 subject, in accordance with an exemplary embodiment of the present invention.

FIG. 17B shows correlation and Bland-Altman plots for mean arterial pressure (MAP), diastolic pressure (DP), and systolic pressure (SP) for a study having 21 subjects, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

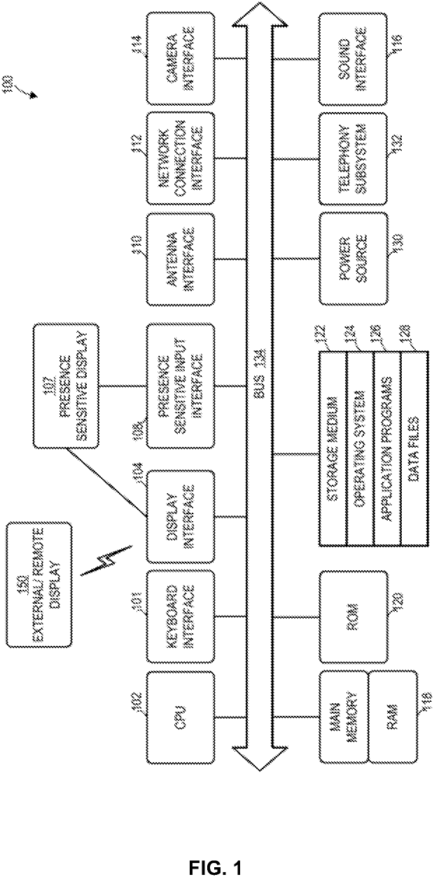

Various systems, methods, and computer-readable mediums are disclosed for conveying chorded systems and will now be described with reference to the accompanying figures. FIG. 1 depicts a block diagram of an illustrative computing device architecture 100, according to an example embodiment. Certain aspects of FIG. 1 may be embodied in a computing device 100. As desired, embodiments of the disclosed technology may include a computing device with more or less of the components illustrated in FIG. 1. It will be understood that the computing device architecture 100 is provided for example purposes only and does not limit the scope of the various embodiments of the present disclosed systems, methods, and computer-readable mediums.

The computing device architecture 100 of FIG. 1 includes a CPU 102, where computer instructions are processed; a display interface 104 that acts as a communication interface and provides functions for rendering video, graphics, images, and texts on the display. In certain embodiments of the disclosed technology, the display interface 104 may be directly connected to a local display, such as a touch-screen display associated with a mobile computing device. In another example embodiment, the display interface 104 may be configured for providing data, images, text, and other information for an external/remote display that is not necessarily physically connected to the mobile computing device. For example, a desktop monitor may be utilized for mirroring graphics and other information that is presented on a mobile computing device. In certain some embodiments, the display interface 104 may wirelessly communicate, for example, via a Wi-Fi channel or other available network connection interface 112 to the external/remote display.

In an example embodiment, the network connection interface 112 may be configured as a communication interface and may provide functions for rendering video, graphics, images, text, other information, or any combination thereof on the display. In one example, a communication interface may include a serial port, a parallel port, a general purpose input and output (GPIO) port, a game port, a universal serial bus (USB), a micro-USB port, a high definition multimedia (HDMI) port, a video port, an audio port, a Bluetooth port, a near-field communication (NFC) port, another like communication interface, or any combination thereof.

The computing device architecture 100 may include a keyboard interface 106 that provides a communication interface to a keyboard. In one example embodiment, the computing device architecture 100 may include a presence-sensitive display interface 107 for connecting to a presence-sensitive display. According to certain some embodiments of the disclosed technology, the presence-sensitive display interface 107 may provide a communication interface to various devices such as a pointing device, a touch screen, a depth camera, etc. which may or may not be associated with a display.

The computing device architecture 100 may be configured to use an input device via one or more of input/output interfaces (for example, the keyboard interface 106, the display interface 104, the presence sensitive display interface 107, network connection interface 112, camera interface 114, sound interface 116, etc.) to allow a user to capture information into the computing device architecture 100. The input device may include a mouse, a trackball, a directional pad, a track pad, a touch-verified track pad, a presence-sensitive track pad, a presence-sensitive display, a scroll wheel, a digital camera, a digital video camera, a web camera, a microphone, a sensor, a smartcard, and the like. Additionally, the input device may be integrated with the computing device architecture 100 or may be a separate device. For example, the input device may be an accelerometer, a magnetometer, a digital camera, a microphone, and an optical sensor.

Example embodiments of the computing device architecture 100 may include an antenna interface 110 that provides a communication interface to an antenna; a network connection interface 112 that provides a communication interface to a network. In certain embodiments, a camera interface 114 is provided that acts as a communication interface and provides functions for capturing digital images from a camera. In certain embodiments, a sound interface 116 is provided as a communication interface for converting sound into electrical signals using a microphone and for converting electrical signals into sound using a speaker. According to example embodiments, a random-access memory (RAM) 118 is provided, where computer instructions and data may be stored in a volatile memory device for processing by the CPU 102.

According to an example embodiment, the computing device architecture 100 includes a read-only memory (ROM) 120 where invariant low-level system code or data for basic system functions such as basic input and output (I/O), startup, or reception of keystrokes from a keyboard are stored in a non-volatile memory device. According to an example embodiment, the computing device architecture 100 includes a storage medium 122 or other suitable type of memory (e.g., RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives), where the files include an operating system 124, application programs 126 (including, for example, a web browser application, a widget or gadget engine, and or other applications, as necessary) and data files 128 are stored. According to an example embodiment, the computing device architecture 100 includes a power source 130 that provides an appropriate alternating current (AC) or direct current (DC) to power components. According to an example embodiment, the computing device architecture 100 includes a telephony subsystem 132 that allows the transmission and receipt of sound over a telephone network. The constituent devices and the CPU 102 communicate with each other over a bus 134.

According to an example embodiment, the CPU 102 has appropriate structure to be a computer processor. In one arrangement, the CPU 102 may include more than one processing unit. The RAM 118 interfaces with the computer bus 134 to provide quick RAM storage to the CPU 102 during the execution of software programs such as the operating system application programs, and device drivers. More specifically, the CPU 102 loads computer-executable process steps from the storage medium 122 or other media into a field of the RAM 118 in order to execute software programs. Data may be stored in the RAM 118, where the data may be accessed by the computer CPU 102 during execution. In one example configuration, the device architecture 100 includes at least 125 MB of RAM, and 256 MB of flash memory.

The storage medium 122 itself may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DVD) optical disc drive, an internal hard disk drive, a Blu-Ray optical disc drive, or a Holographic Digital Data Storage (HDDS) optical disc drive, an external mini-dual in-line memory module (DIMM) synchronous dynamic random access memory (SDRAM), or an external micro-DIMM SDRAM. Such computer readable storage media allow a computing device to access computer-executable process steps, application programs and the like, stored on removable and non-removable memory media, to off-load data from the device or to upload data onto the device. A computer program product, such as one utilizing a communication system may be tangibly embodied in storage medium 122, which may comprise a machine-readable storage medium.

According to one example embodiment, the term computing device, as used herein, may be a CPU, or conceptualized as a CPU (for example, the CPU 102 of FIG. 1). In this example embodiment, the computing device may be coupled, connected, and/or in communication with one or more peripheral devices, such as display. In this example embodiment, the computing device may output content to its local display and/or speaker(s). In another example embodiment, the computing device may output content to an external display device (e.g., over Wi-Fi) such as a TV or an external computing system.

In some embodiments of the disclosed technology, the computing device 100 may include any number of hardware and/or software applications that are executed to facilitate any of the operations. In some embodiments, one or more I/O interfaces may facilitate communication between the computing device and one or more input/output devices. For example, a universal serial bus port, a serial port, a disk drive, a CD-ROM drive, and/or one or more user interface devices, such as a display, keyboard, keypad, mouse, control panel, touch screen display, microphone, etc., may facilitate user interaction with the computing device. The one or more I/O interfaces may be utilized to receive or collect data and/or user instructions from a wide variety of input devices. Received data may be processed by one or more computer processors as desired in various embodiments of the disclosed technology and/or stored in one or more memory devices.

One or more network interfaces may facilitate connection of the computing device inputs and outputs to one or more suitable networks and/or connections; for example, the connections that facilitate communication with any number of sensors associated with the system. The one or more network interfaces may further facilitate connection to one or more suitable networks; for example, a local area network, a wide area network, the Internet, a cellular network, a radio frequency network, a Bluetooth enabled network, a Wi-Fi enabled network, a satellite-based network any wired network, any wireless network, etc., for communication with external devices and/or systems.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

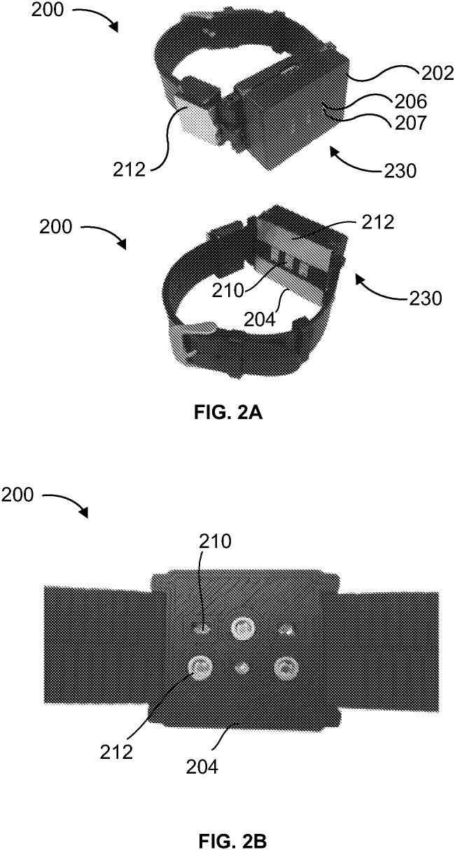

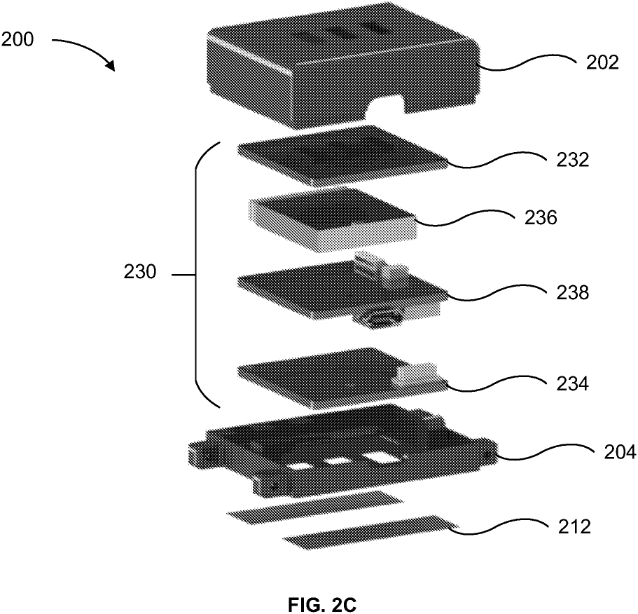

As shown in FIG. 2A, an exemplary embodiment of the present disclosure provides a device 200 for non-invasively measuring blood pressure in a subject. As would be appreciated by those of skill in the relevant art, the method can be implemented into any device or system that is capable of having two or more sensors, as described herein.

In some embodiments, a device can be any suitable type of device used portably, at home, or on the go, provided that the device has two or more sensors. In some embodiments, the device can be temporarily contacted to the subject’s body, but not attached, in order to detect signals and estimate a blood pressure (BP) measurement. For instance, a phone sensor can be contacted to the subject’s body such that the phone sensor can detect one or more signals and perform the methods for measuring BP, as described herein. Other devices that can temporarily contact a subject’s body include, but are not limited to, a stethoscope, a scale, a sensor pad, a pulsometer, etc. In general, most conventional devices having sensors would require two or more sensors, either in the same device or in communication with another device, in order to estimate a blood pressure measurement.

In some embodiments, a device for measuring blood pressure can be an accessory (e.g., watches, glasses, heart rate monitors, a patch adhered to the skin, belts, shoes, etc.,), clothing (e.g., vests, socks, shirts, etc.,), jewelry (e.g., rings, necklaces, earrings, bracelets, etc.,), and/or medical equipment that has been embedded or integrated with sensors to generate “smart” wearable devices. As would be appreciated by those of skill in the art, any suitable type of device that can be embedded with sensors that is also wearable by a subject, such as a human or an animal, can implement the method for estimating BP as disclosed herein.

Referring back to FIG. 2A, a wearable device 200 can have a first surface 202. In some embodiments, wearable device 200 can be custom made into any shape and/or dimension such that appropriate contact between first surface 202 and the subject can be adequately acquired. Additionally, wearable device 200 can be made of a variety of materials, such as, for example, paper, glass, metal, steel, stainless steel, concrete, wood, aluminum, copper, iron, electronics, plastic, textiles, ceramic, plaster, leather, stone, cork, and the like.

In some embodiments, wearable device 200 can have a second surface 204. Second surface 204 can be an opposing surface to first surface 202 but is not constricted to such a design. For example, second surface 204 can be adjacent to, aligned with, axial, concentric with, contiguous, extended from, overlapping to, perpendicular of, parallel to, retractable from, staggered, surrounding, and/or juxtaposed to first surface 202. In some embodiments, wearable device 200 can have additional surfaces other than first and second surfaces 202, 204. As would be understood by one of skill in the art, first surface 202 and second surface 204 can be interchanged with respect to orientation or placement on wearable device 200 such that first surface 202 can be contacting the subject’s body while second surface 204 can be outward facing; however it is not to be limited to such an orientation (e.g., first surface 202 can be contacting the subject’s body in one location while second surface 204 can be contacting the subject’s body in a different location).

In any of the embodiments disclosed herein, wearable device 200 can have a first sensor 206 positioned on first surface 202, such that first sensor 206 can receive and/or detect one or more signals external to wearable device 200. First sensor 206 can include any suitable apparatus or combination of suitable apparatuses for detecting and/or receiving light (e.g., photoresistors, light-dependent resistors, photodiodes, photosensors, photodetectors, etc.,). In some embodiments, the light received is a mirrored or reflected light from an actuator 207 configured to emit a measuring signal. First sensor 206 can be configured to receive one or more wavelengths of light, ranging from infrared (about 2000 nm) to deep ultraviolet (about 100 nm). Actuator 207 can include any apparatus or combination of apparatuses for emitting wavelengths of light. For example, actuator 207 can be one or more optical measurement apparatuses such as a light source, (e.g., light emitting diodes (LEDs), incandescent light, luminescent light, etc.,). The light source can emit light in wavelengths ranging from infrared (about 2000 nm) to deep ultraviolet (about 100 nm). In some embodiments, first sensor 206 and actuator 207 may be combined into a single chip having one or more light sources combined with one or more photodetectors. In any of the embodiments herein, first sensor 206 can be any photoplethysmography (PPG)-based monitoring apparatus. First sensor 206 can be a suitable non-invasive means for measuring volumetric variations of blood circulation at the surface of skin and the arterioles of a subject. In general, a typical PPG sensor can contain a light source for emitting light to a tissue. The typical PPG sensor can also contain a photodetector for measuring the reflected light from the tissue. The reflected light is proportional to blood volume variations and can be used measure heart rate.

In some embodiments, first sensor 206 can be a PPG apparatus with one or more lights sources having one or more distinct wavelengths. In certain embodiments, first sensor 206 can emit an infrared LED and a green LED. An infrared LED can be used to measure the flow of blood that is more deeply concentrated in certain parts of the body, such as, for example, deeper than peripheral vessels, in muscles, or below fat. A red LED, independently or in combination with an infrared LED can be used to measure and calculate the absorption of oxygen in peripheral vessels while a green LED can measure absorption of oxygen closer to the surface of the skin and is primarily used to detect and/or monitor heart rate. Additional wavelength LEDs (e.g., red, orange, yellow, blue, violet, ultraviolet, etc.,) can be implemented to measure blood volume changes in vessels of varying depths. In some embodiments, first sensor 206 can be a PPG apparatus with one or more photodetectors for detecting reflected light from each light source. In an example wearable device having three PPG sensors with three different LEDs positioned on first surface 202, one of the photodetectors can be configured to block two of the LEDs, such as the red LED and the infrared LED wavelengths, so that the detection of the third LED by one of the photodetectors can be improved. In embodiments having three LEDs and two photodetectors, one photodetector can have peak sensitivity for one of the LEDs while the other photodetector can have enhanced sensitivity for the two other LEDs. It is contemplated that first sensor 206 can have three or more LEDs and three or more photodetectors, wherein each LED has a respective photodetector with enhanced sensitivity for the LED’s emitted wavelength.

In any of the embodiments disclosed herein, wearable device 200 can include one or more PPG sensors positioned on first surface 202. First surface 202 can include a slot for each PPG sensor such that each PPG sensor can emit and/or detect signals at the surface of a subject skin. In some embodiments, three slots can be made on first surface 202 such that the three PPG sensors can be placed on first surface 202 to emit light and detect reflected light through the three slots. While in use, the three PPG sensors can be positioned over a first vessel and can be configured to measure a first blood volume change within the first vessel. In some embodiments, the first vessel can be any subcutaneous blood vessel, including arterioles and peripheral vessels (e.g., blood vessels of the ear, nasal septum, forehead, sternum, fingertip, wrist, toe, foot, and the like). In some embodiments, first sensor 206 can be configured to detect a first signal. The first signal can include a first PPG signal from any subcutaneous blood vessel, from which a variety of PPG feature points can be identified (e.g., systolic peak, dicrotic notch, diastolic foot, maximum slop, maximum concavity, systolic amplitude, pulse width, pulse area, peak to peak interval, pulse interval, augmentation index, large artery stiffness index.)

In any of the embodiments disclosed herein, wearable device 200 can also include a second sensor 208 positioned within wearable device 200 and/or on a surface of wearable device 200, such as first surface 202, second surface 204, or any other surface. Second sensor 208 can be configured to detect one or more signals external to wearable device 200. Second sensor 208 can include any apparatus or combination of apparatuses for detecting motion (e.g., rotary, oscillating, linear, and/or reciprocating motion). For example, second sensor 208 can be one or more accelerometer, magnetometer, digital camera, microphone, ultrasonic sensor, microwave sensor, and/or optical sensor for detecting motion and/or activity in a subject. Second sensor 208 apparatuses can include, but are not limited to, uniaxial and/or triaxial pizoelectric accelerometers, MEMS accelerometers, smartphone accelerometers and gyroscopes, triaxial gyroscopes, laser Doppler vibrometers, microwave Doppler radars, airborne ultrasound surface motion cameras, and the like.

In any of the embodiments herein, second sensor 208 can be configured to detect and measure cardiac mechanical vibrations. Second sensor 208 can be any seismocardiograph (SCG)-based apparatus. SCG-based sensors can detect cardiac mechanical vibrations useful for generating one or more SCG signals, from which a variety of SCG feature points and cardiac time intervals can be identified (e.g., peak of atrial systole (AS), mitral valve closure (MC), peak of rapid systolic ejection (RE), peak of rapid diastolic filling (RF), isovolumic contraction (IC), mitral valve opening (MO), aortic valve closure (AC), aortic valve opening (AO), isovolumic movement (IM), rapid diastolic filling time, isotonic contraction (IC). isovolumic relaxation time (IVRT), left ventricular ejection time (LVET), maximum acceleration in aorta (MA), pre-ejection period (PEP), total electromechanical systole period (QS2), maximum blood injection (MI), isovolumic contraction time (IVCT), left ventricular lateral wall contraction peak velocity (LCV), septal wall contraction peak velocity (SCV), trans-aortic peak flow (AF), trans-pulmonary peak flow (PF), trans-mitral ventricular relaxation flow (MF_{E}), and atrial contraction flow (MF_{A})).

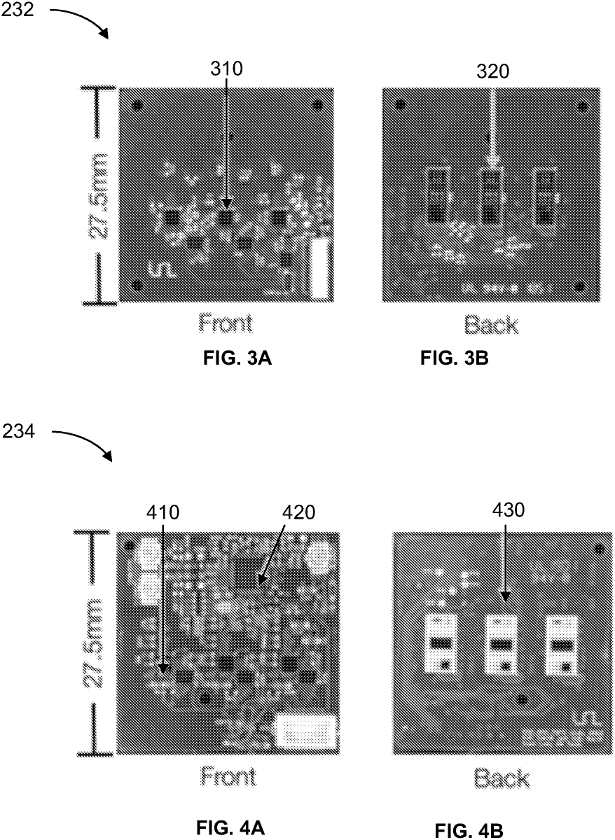

As shown in FIG. 2C, wearable device 200 can include a processor 230 positioned within wearable device 200. Processor 230 can include one or more PPG boards, batteries, and main boards. In some embodiments, processor 230 can have a first PPG board 232 positioned near and/or adjacent to first surface 202 such that first PPG board 232 can be used to receive a PPG signal from a blood vessel contacting first surface 202. As shown in FIGS. 3A and 3B, PPG board 232 can have a PPG analog front-end (AFE) 310 and one or more light sources and photodetector pairs 320. In some embodiments, processor 230 can be configured to receive PPG signals from first PPG board 232 and second PPG board 234 at or about the same time.

In some embodiments, processor 230 can include a second PPG board 234 positioned near and/or adjacent to second surface 204 such that second PPG board 234 can be used to receive a PPG signal from a blood vessel contacting second surface 204. As shown in FIGS. 4A and 4B, second PPG board 234 can be a combination of PPG and ECG and can have a PPG AFE 410, an ECG 420 for electrical biosensing, and one or more light sources and photodetector pairs 430.

In some embodiments, processor 230 can include a battery 236 and main board 238. It should be understood that processor 230 can include one or more batteries as well as one or more main boards. Mainboard 238 can include an SD card 510, a microcontroller 520, an environmental sensor 530, an accelerometer 540, a gyroscope, 550, a charging circuit 560, and a connector 570 connecting mainboard 238 to second PPG board 234.

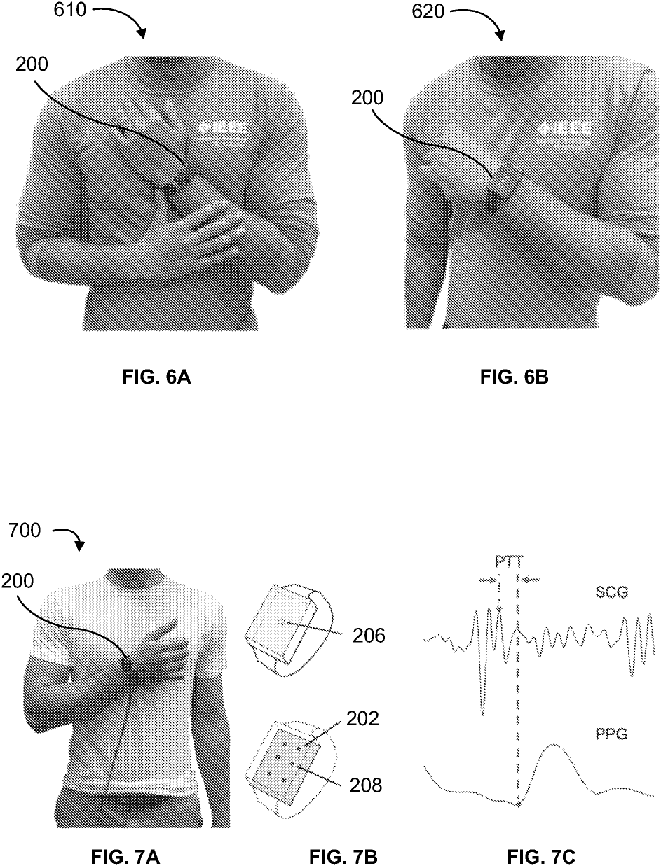

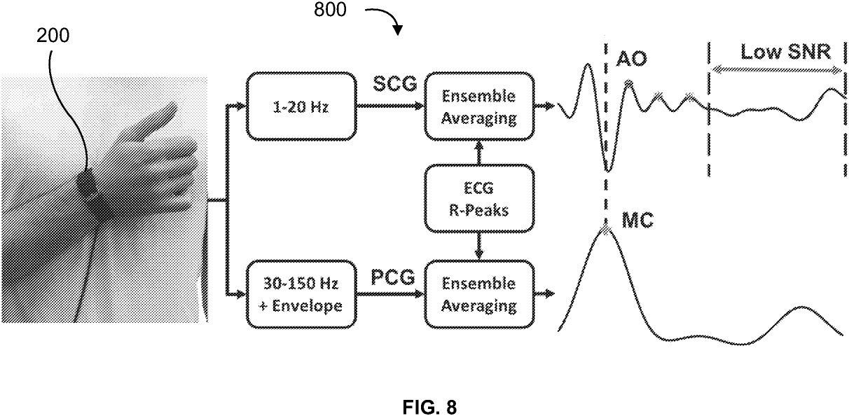

As shown in FIGS. 6A and 6B, processor 230 can generate output representing a blood pressure measurement of a subject based on a PPG signal from first sensor 206 positioned on first surface 202 and an SCG signal from second sensor 208 positioned within wearable device 200. As shown in FIGS. 7A-7C, when first surface 202 of wearable device 200 is positioned over a first vessel, such as peripheral blood vessels of a subject’s sternum, first sensor 206 (connected to first PPG board 232 within processor 230) can receive a first PPG signal from the subject’s sternum while second sensor 208 (connected to mainboard 238 within processor 230) can simultaneously receive a SCG signal from the subject’s cardiac mechanical vibrations. As shown in FIG. 7C, from a PPG signal and an SCG signal, a pulse transit time (PTT) can be determined. FIG. 8 shows a block diagram of an example signal processing technique for extracting the aortic opening (AO) peak from second sensor 208 when filtering bandwidths (a) from about 0.8 Hz to about 30 Hz to receive the SCG signal and (b) from about 30 Hz to about 150 Hz to receive a phonocardiogram (PCG) signal.

In any of the embodiments disclosed herein, and as shown in FIG. 2B, wearable device 200 can also include a third sensor 210. Third sensor 210 can be positioned on second surface 204 of wearable device 200. In some embodiments, third sensor 210 can be positioned on any surface of wearable device 200 that is spatially separated from first sensor 206. Third sensor 210 can include a second PPG-based sensor, substantially similar to first sensor 206 and actuator 207, as described above. In certain embodiments, third sensor 210 can be a second PPG-based sensor that differs from first sensor 206. As shown in FIG. 3 and FIG. 4, first sensor 206 can be connected to first PPG board 232 and have PPG AFE 310 and one or more pairs of light sources and photodetectors 320, while third sensor 210 can be connected to second PPG board 234 have PPG AFE 410, an ECG 420 for electrical biosensing, and one or more pairs of light sources and photodetectors 430.

In some embodiments, adding third sensor 210 to wearable device 200 to receive a second PPG-based signal may allow for additional timing references for a resulting PPG signal and can perform motion artifact cancellation such that a reliable blood pressure measurement may be estimated for a subject. Additionally, a second PPG signal from a multi-wavelength first sensor 206 and third sensor 210 may provide optimal PPG signals based on contact with ideal peripheral blood vessels to produce a high-fidelity PTT extraction.

In an example embodiment, as shown in FIGS. 6–8, wearable device 200 can be configured to be worn around a subject’s wrist similar to a wristwatch, with first surface 202 positioned on the watch face and configured to be positioned against the subject’s sternum. First sensor 206 can be configured to measure a PPG signal from the sternum’s pulse wave using first PPG board 232 while second sensor 208 can simultaneously measure a SCG signal from the sternum’s cardiac mechanical vibrations. Second surface 204 can be positioned to be closer to the wrist when worn. Third sensor 210 positioned on second surface 204 can be configured to measure a second PPG signal from the subject’s wrist, using second PPG board 234.

Referring back to FIG. 2A, wearable device 200 can further comprise a fourth sensor 212, such that fourth sensor 212 can receive one or more electrical activities of the subject through one or more electrode and actuator pairs. Fourth sensor 212 can be positioned on second surface 204, as shown in FIG. 2C, or can be positioned on any additional surface of wearable device 200, as shown in FIG. 2A (top). Fourth sensor 212 can include any apparatus or combination of apparatuses for receiving electrical signals. For example, fourth sensor 212 can include one or more wet electrodes or dry stainless-steel electrodes. In some embodiments, fourth sensor 212 can be positioned on second surface 204 such that one or more electrodes can contact subject’s wrist. In certain embodiments, fourth sensor 212 can be positioned on an additional surface of wearable device 200 such that the subject can contact a portion of subject’s body to one or more electrodes. For instance, when fourth sensor 212 is positioned on a wristwatch strap facing outward from the subject’s wrist, the subject can contact a thumb from the opposite hand to fourth sensor 212 to allow for an electrical signal measurement independently or in combination with first, second, and/or third sensors. Fourth sensor 212 can be configured to receive an electrocardiogram (ECG) signal, an impedance cardiogram (ICG) signal, or an impedance plethysmogram (IPG) signal. Fourth sensor 212 can be any electrocardiogram (ECG)-based sensor that can allow for easy partitioning of a subject’s heart beats and can be used to assess the heart rate variability (HRV) and determine autonomic state. Fourth sensor 212 can be a pair of electrodes that are configured to sense electrical activity and a pair of electrodes that are configured to emit and/or input current. In general, fourth sensor 212 can include a pair of electrodes and a pair of actuators configured to input current to the body.

In any of the embodiments disclosed herein, wearable device 200 can include one or more ECG sensors. As depicted above and shown in FIG. 4A, second PPG board 234 may combine PPG and ECG sensors for independently and/or simultaneously receiving PPG feature points and ECG feature points. ECG feature points can include, but are not limited to, the detection of the R-wave peak, the area under the curve, peak amplitude, time delay between peaks and valleys, absolute timing of peaks, and heart rate frequency. In some embodiments, wearable device 200 can include a fifth sensor for receiving one of an ECG signal, a ICG signal, or an IPG signal. The fifth sensor can connect to the electrical apparatus or combination of electrical apparatuses from fourth sensor 212.

In some embodiments, wearable device 200 can also include a sixth sensor 550 positioned within wearable device 200, such as on mainboard 238. Sixth sensor 550 can be configured to measure motion (e.g., rotary, oscillating, linear, and/or reciprocating motion) and/or orientation. Sixth sensor 550 can be configured to receive a gyrocardiogram (GCG) signal. For example, sixth sensor 550 can be one or more of a spinning mass gyroscope, mechanical gyroscope, gas-bearing gyroscope, vibrating structure gyroscope, optical gyroscope, MEMS gyroscope, and the like.

In any of the embodiments disclosed herein, wearable device 200 can also include one or more environmental sensors 530 positioned within wearable 200, such as on mainboard 238. Environmental sensors 530 can be configured to provide detailed and/or reliable data regarding environmental parameters, such as, for example, humidity, temperature, barometric pressure, noise, carbon dioxide concentration, volatile organic compound (VOC) concentration, and the like.

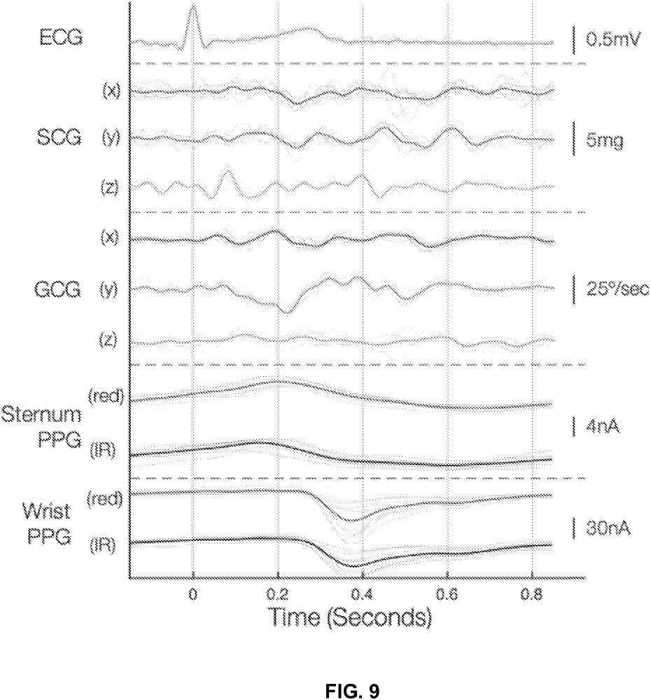

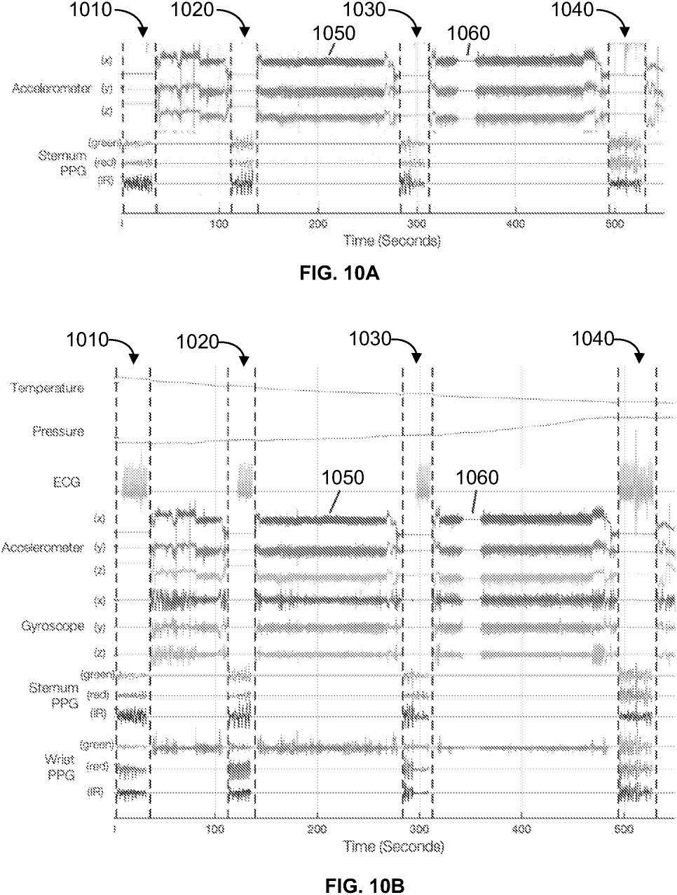

As shown in FIG. 9, systems and methods of the present disclosure can be configured to generate one or more signals independently and/or simultaneously over a period of time. Wearable device 200 can receive a first PPG signal from a subject’s sternum, an SCG signal from the subject’s sternum, a second PPG signal, the second PPG signal coming from the subject’s wrist, and ECG signal from contact with the subject’s skin at the wrist, and a GCG signal. In some embodiments, wearable device 200 can take a measurement when a subject places a finger or thumb on an electrical sensor on a surface of wearable device, as shown in FIG. 6A. In other embodiments, wearable device 200 can take measurements during a subject’s activity, such as during a walk. Specifically, FIG. 10A shows a measurement based on a PPG signal from first sensor 206 and a SCG signal from second sensor 208 when a subject is sitting at a desk 1010, standing outside 1020, standing at the top of a hill 2030, standing at the bottom of a hill 2040, walking 2050, and standing at a traffic light 1060. FIG. 10B shows measurements received from a combination of first PPG signal from first sensor 206, SCG signal from second sensor 208, second PPG signal from third sensor 210, ECG signal from fourth signal 212, GCG signal from sixth sensor 550, and temperature and pressure signals from environmental sensors 530.

In some embodiments, measurements received based on one or more PPG signals, SCG signals, ECG signals, and GCG signals can correlate to one or more hemodynamic variables, including, but not limited to, pulse transit time (PTT), pulse arrival time (PAT), pre-ejection period (PEP), blood pressure (BP), pulse wave velocity (PWV), arterial stiffness and the like. To estimate PTT, either the first PPG signal or the second PPG signal can be combined with the SCG signal. In some embodiments, PTT can be estimated using multi-site PPG using first sensor 206 and third sensor 210 in combination with second sensor 208 of wearable device 200. To estimate PAT, either the first PPG signal or the second PPG signal can be combined with the ECG signal. In some embodiments, PAT can be estimated using multi-site PPG using first sensor 206 and third sensor 210 in combination with fourth sensor 212 or the fifth sensor of wearable device 200. To estimate PEP, the SCG signal can be combined with the ECG signal. In some embodiments, PEP can be estimated using second sensor 208 in combination with fourth sensor 212 or the fifth sensor of wearable device 200. As would be appreciated by those of skill in the art, additional sensors can be included when estimating the PTT, PAT, or PEP in order to generate a more reliable estimation.

In any of the embodiments disclosed herein, a blood pressure measurement can be extracted from the one or more hemodynamic variables described above. In some embodiments, a BP measurement extracted from PTT can be more reliable than PEP during certain conditions, such as ruing exercise recovery.

An exemplary embodiment of the present disclosure provides processor 230 configured to transition wearable device 200 from a normal mode of operation to one or more measurement modes of operation. In some embodiments, transition between modes can be manually triggered by the subject. As shown in FIG. 6A, the subject can place a finger or thumb on an electrical sensor on a surface of wearable device 200 to initiate processor 230 to transition wearable device 200 from a normal mode to a measurement mode. In any of the embodiments herein, transition between modes can be automatic. Wearable device 200 can be configured to initiate processor 230 to automatically transition to a measurement mode when a subject positions wearable device 200 on or near the subject’s chest. Alternatively, wearable device 200 can be configured to detect electrical activity near fourth sensor 212 or fifth sensor such that processor 230 can transition to measurement mode when a weak electrical activity is detected. When wearable device 200 comprises a wristwatch, additional automatic triggers and/or pressure sensors can include, but are not limited to patterned motions of the subject, periods likely to induce large changes in blood pressure such as postural shifts and/or cessation of exercise, covering the wristwatch face completely, and the like.

During standby mode, processor 230 can be configured to shut down all sensors on wearable device 200 except for a single wavelength of one of the PPG sensors. For example, a single green PPG sensor on second surface 204, contacting the subject’s wrist can be configured to act as a proximity sensor. In some embodiments, standby mode can conserve battery power, for example, the single green PPG sensor can be sampled at about 8 Hz or less.

In some embodiments, wearable device 200 can detect an automatic trigger and processor 230 can be configured to transition into one or more measurement modes. For example, when an object approaches a sensor, such as when the subject places the wearable device 200 on the wrist, processor 230 can be configured to transition to continuous mode.

In some embodiments, processor 230 can be configured to monitor certain signals. For instance, in continuous mode, processor 230 can be configured to initiate three green wrist PPG sensors, the SCG sensor, and the GCG sensor. In some embodiments, the environmental sensor can also be initiated, and sampled at 125 Hz. The lower sample rate saves power while providing enough context for activity classification. In some embodiments, the normal mode can include detection of a wrist PPG signal, a SCG signal, and an ECG signal. In such an example, wearable device 200 is configured to monitor hemodynamic variables such as heart rate.

In some embodiments, processor 230 can be configured to transition to PTT, PAT, or PEP measurement mode when the ECG sensor detects a manual or an automatic trigger. In some embodiments, the subject can touch an electrical sensor positioned on one of the surfaces, such as the wrist-band.

In some embodiments, processor 230 can be configured to initiate two or more sensors while in measurement mode. For example, in PTT measurement mode, processor 230 can be configured to initiate the sternum PPG sensors and the SCG sensor while wearable device 200 is placed directly in contact with the subject’s skin at the mid sternum. In some embodiments, in PTT measurement mode, processor 230 may optionally be configured to also initiate wavelengths (i.e., green, red, and IR) of both the wrist PPG sensor.

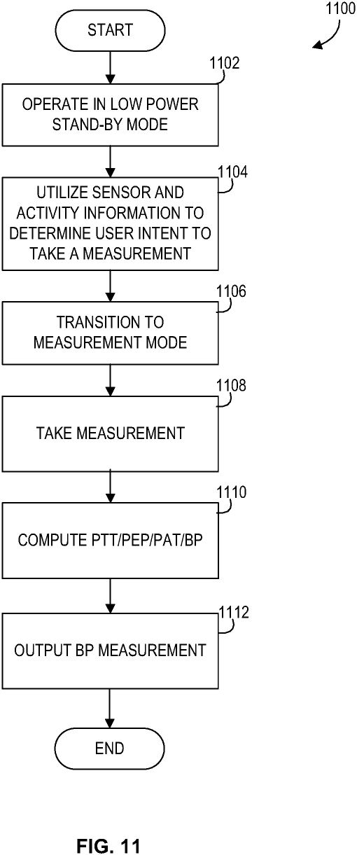

As shown in FIG. 11, processor 230 can be configured to transition between modes in order to estimate a BP measurement. The method 1100 for transition can include the steps of operating in low power stand-by mode 1102, utilizing sensor information and/or activity information in order to determine user intent to take a measurement 1104, transitioning to measurement mode 1106, taking measurement 1108, computing PTT, PEP, PAT, and/or BP 1110, and outputting a BP measurement 1112.

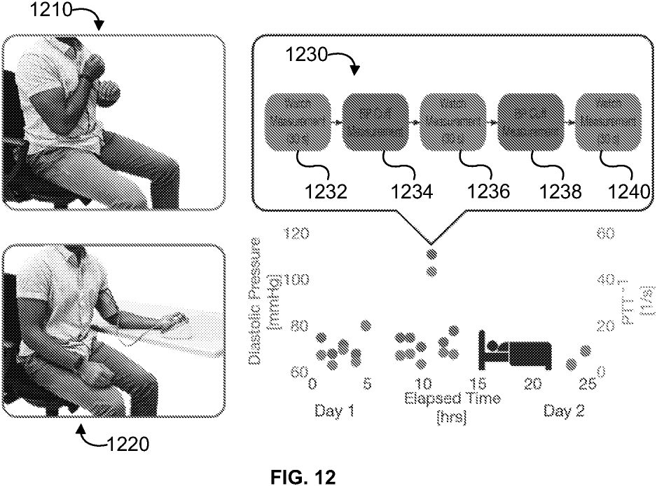

As shown in FIG. 12, wearable device 200 can be configured to transition to measurement mode in order to calibrate measurements for each individual subject. In some embodiments, wearable device 200 can be calibrated by acquiring pulse transit time (PTT) and blood pressure (BP) cuff measurements hourly over the course of 24 hours, except during sleep. Each measurement can include a sequence of PTT measurements, acquired by contacting wearable device 200 to the subject’s sternum 1210, and a sequence of BP cuff measurements, taken using an oscillometric cuff 1220. The measurement sequence 1230 can include a first wearable device measurement 1232 and a first BP cuff measurement 1234 followed by a second wearable device measurement 1236 and a second BP cuff measurement 1238.

In any embodiment disclosed herein, wearable device 200 can be configured to provide an alert to the subject that a measurement should be collected in order to generate a calibration. In general, a trend in blood pressure can be estimated from a calibration curve having at least one measurement point when the subject was experiencing high blood pressure and at least one measurement point when the subject was experiencing low blood pressure. In some embodiments, wearable device 200 can be configured to alert the subject when one or more detected hemodynamic variables indicates an opportune time to transition into measurement mode. For example, when wearable device 200 detects an increased heart rate while in continuous mode, processor 230 may be configured to alert the subject (e.g., electrical or vibrational stimulation) to initiate one of the measurement modes to add a measurement point to a calibration curve.

In some embodiments, the calibration curve generated by wearable device 200 from two or more measurement points collected can produce a trend from fewer measurement points than any conventional method. As would be appreciated by those of skill in the art, collecting a blood pressure measurement when a subject’s other hemodynamic variables (e.g., heart rate) indicates the subject may be stressed or anxious, could verify the blood pressure calibration curve and assist in monitoring the subject’s overall blood pressure health.

In some embodiments, the calibration curve generated by wearable device 200 can be configured to incorporate parameters estimated for a specific subject such that the calibration curve is a hybrid individualized calibration. Example parameters incorporated can include, but are not limited to, cardiovascular risk factors (e.g., diabetes mellitus, hypertension, overweight (BMI > 25 kg/m², family history of heart failure, past chemotherapy, or antihypertensive medication), measurements from oscillometric devices, and the like. A subject can input specific parameters such that the calibration curve generated by wearable device 200 can factor in additional individualized variables into the blood pressure trend created from receiving blood pressure measurements.

In certain embodiments, the calibration curve generated by wearable device 200 can incorporate parameters that are derived from the general population. For example, the incorporated parameters can include data from epidemiological and/or population statistics such that the subject’s blood pressure trend can be a based substantially on a general population trend. In yet another embodiment, the calibration curve can be generated by a combination of blood pressure measurements received by wearable device 200 and one or more parameters derived from demographic, epidemiological and/or population statistics. For example, the subject’s blood pressure trend can be based on the individual blood pressure measurements from wearable device 200 in combination with a population-based trend.

The following examples further illustrate aspects of the present disclosure. However, they are in no way a limitation of the teachings or disclosure of the present disclosure as set forth herein.

### EXAMPLES

### Example 1: Recording Seismocardiogram and Photoplethysmogram Signals in a Wristwatch Form Factor for PTT Measurements

The method uses a wristwatch form factor, similar to that of fitness monitoring wearable devices currently in the market (e.g., Fitbit, Apple Watch, etc.). The methods described herein measure PTT when the user places the face of the watch onto the sternum for a short period of time (< 15 seconds). An accelerometer inside the watch measures the SCG for the proximal timing reference, and PPG sensors facing the wrist measure the distal timing reference. Though the required user input is not conducive for continuous measurements, the device can provide episodic BP estimation as a more convenient alternative to the conventional BP oscillometric cuffs.

### Example 2: Hardware Design

The system focuses on obtaining SCG and PPG measurements to simultaneously detect both the proximal and distal timing reference, respectively. This system was able to obtain, for the first time, both references from the same convenient wearable device and can be used in an at-home setting by performing a simple maneuver. All parts are commercially available, with similar devices being used in current smart watches.

To measure the SCG the ADXL354 accelerometer (Analog Devices, Norwood, MA) was chosen due to its ultra-low noise floor

$$\left(20 μ g / \sqrt{H z}\right) .$$

A low noise floor accelerometer was needed since the SCG signal amplitude is small, typically around 10 mg to 50 mg peak to peak. The Apple Watch (Apple Inc, Cupertino, CA) uses the BMA 280 (Bosch, Stuttgart, Germany) with a noise level of

$$120 μ g / \sqrt{H z}$$

or an average peak to peak of 5 mg peak to peak. This noise would couple into the SCG and make feature extraction difficult. The ADXL354 limits the noise to only 0.8 mg peak to peak and reduces the noise in the SCG signal.

The systems described herein use an array of three IR LED and photodiode pairs. While most wrist-based heart rate monitors (including the Apple Watch) use green LEDs to maximize signal quality for heart rate extraction, IR LEDs allowed for deeper penetration into the skin and for the capture of the arterial pulse wave from the larger arteries. The cathode of the photodiode was biased to 5 V to increase the sensor’s linearity and the anodes were connected to a transimpedance amplifier configured to act as a first-order low-pass filter (f_{χ} = Hz, G = 110 dB) followed by a band-pass filter (BW = 0.7 - 8 Hz, G = 20 dB). A potentiometer in series with the LEDs allowed for manual subject-specific calibration of the LEDs light intensity. After visual inspection of the signal during the study, the light intensity was altered, accounting for differing melanin levels or arterial depths.

A custom 3D printed watch was designed to house the accelerometer, photodiodes, and IR LEDs. The watch was tethered to an external box that housed the power supply and AFEs. The backside of the watch that made contact with the wrist included three cutouts approximately 1 cm apart to expose the pairs of photodiodes and LEDs. The spacing allowed for adequate coverage of the wrist and increased the chance of sensing the radial artery.

### Example 3: Human Subject Studies

To obtain a PTT measurement, the user first rotated the watch to the anterior portion of the wrist, ideally toward the lateral side. This positioned the LEDs above the radial artery. The user then placed the top face of the watch on the body of the sternum, above the xiphoid process. In this position, the accelerometer measured the low-frequency thoracic vibrations represented by the SCG while the PPG measured the pulse wave at the wrist. Consequently, the maneuver increased the contact pressure of the PPGs, improving the coupling between the PPGs and skin and increasing the signal quality.

Multiple studies were run to test the robustness and accuracy of the prototype. All studies were approved by IRB, and all subjects provided written informed consent before any studies were begun. In total, thirteen young and healthy subjects were recruited with no history of cardiovascular diseases (age: 23 ± 3; gender: 8 males, 5 females; weight: 68 ± 16 kg; height: 173 ± 10 cm). To acquire a timing reference for the start of a cardiac cycle, the subjects wore standard gel electrodes and a wireless ECG module (BNEL50, Biopack Systems) to measure Lead II ECG. A finger-cuff BP sensor (ccNexfin, Edwards Lifesciences) placed on the hand contralateral to the watch acquired continuous beat-by-beat BP. An MP150 Data Acquisition system (Biopac Systems) sampled all sensors at 2 kHz and stored the data to a desktop computer for post-processing.

Three studies were performed to test the device. For all the studies, the subject were asked to remove coats or other bulky clothing. The first study established a proof-of-concept and an ideal-case correlation between PTT and BP with all thirteen subjects participating. This protocol consisted of three sections: one-minute rest, one-minute exercise, and five-minute recovery. During the rest and recovery periods, the subject sat in a chair with the ccNexfin placed on the left index finger and the watch on the right wrist. During the exercise portion, the watch was removed to prevent damages, and the subject performed a stair stepping exercise.

Eight of the subjects returned for a follow-up study to test the multi-day repeatability of the PTT and BP correlations. This protocol consisted of two sections: one minute-rest and one-minute cold pressor. Cold pressor replaced exercise as the perturbation to test out the correlation curve under conditions that induced different physiological responses. Traditionally, a cold pressor test involves the subject immersing a hand in a bucket of cold water; however, to allow measurements on both hands and to prevent varying localized BP, each subject placed his / her foot in a bucket of cold water.

The final study tested the robustness of the SCG signal on five of the subjects for use in an at-home, unsupervised setting. Contact pressure and sensor location were tested as they are possible sources of user errors that could occur in a home setting. The subjects performed the maneuver and varied the amounts of contact pressures against the sternum, ranging from a light touch to a hard press. Then, the subjects placed the device on varying locations on the chest while maintaining a constant pressure.

### Example 4: Signal Processing

Post-processing techniques on the accelerometer, PPG, and ECG extracted proximal and distal timing references for PTT. Note that the ECG was selected for its high signal quality and simply used in the studies as fiduciary points to validate the other sensor measurements and would not be required in the actual deployment of the device for home use. Similar techniques to the method used for the sole PPG, could extract the beats without the ECG. One of the technical challenges of the SCG signal is the variability in morphology between subjects and its susceptibility to motion artifacts and respiration-induced waveform distortions. Classical models of the SCG denote the AO as the highest peak immediately following the R-wave of the SCG. However, by this simple method, the MC and AO can sometimes be confused with each other even in healthy subjects.

FIG. 8 depicts the method of locating the AO as described herein. To determine the correct waveform, both the SCG and the PCG were utilized from the accelerometer signal. A digital filter (BW = 0.8 Hz - 30 Hz) applied to the accelerometer signal extracted the SCG while a higher band filter (BW = 30 Hz - 150 Hz) extracted the PCG. Note that the PCG in this study was identical to the signal obtained using a digital stethoscope, as the accelerometer acted as a contact microphone. Spline interpolation of the local PCG peaks produced an amplitude envelope. Using the timing of the R-wave, the SCG and the enveloped PCG were partitioned into individual beats and ensemble averaged thirty beats together. Since the PCG is commonly used to identify the closing of the mitral valve, the AO was considered to be the first maximum of the SCG that follows the maximum PCG envelope point. The AO point was assumed to be the maximum that directly followed the MC. This technique can only be used during exercise, due to the shortening of PEP cause the MC and AO to occur in rapid succession. However, PEP should only move the timing of AO point and the waveform following the AO point, including the residual peaks, should be retained. To find the AO point in non-exercise signals, all peaks in the exercise signals were mapped out and the SNR of a 50 ms window around each peak was identified. To find the SNR, the pre-ensembled average beats and the same technique as described above were used. The number of peaks between the AO point and the first peak of low SNR were counted. This count to future SCG beats was applied, counting backward from the first peak with low SNR, to determine the AO.

To extract the foot of the PPG for the distal timing reference, the highest quality PPG signal was manually determined. A digital band-pass filter (BW = 0.8 Hz - 15 Hz) removed out of band noise. An average of thirty beats removed non-periodic noise. The intersecting tangent method then determined the foot of the PPG. The PTT was then simply the difference between the found AO of the SCG and the foot of the PPG.

### Example 5: Assessing Correlations Between Wearable PTT and BP

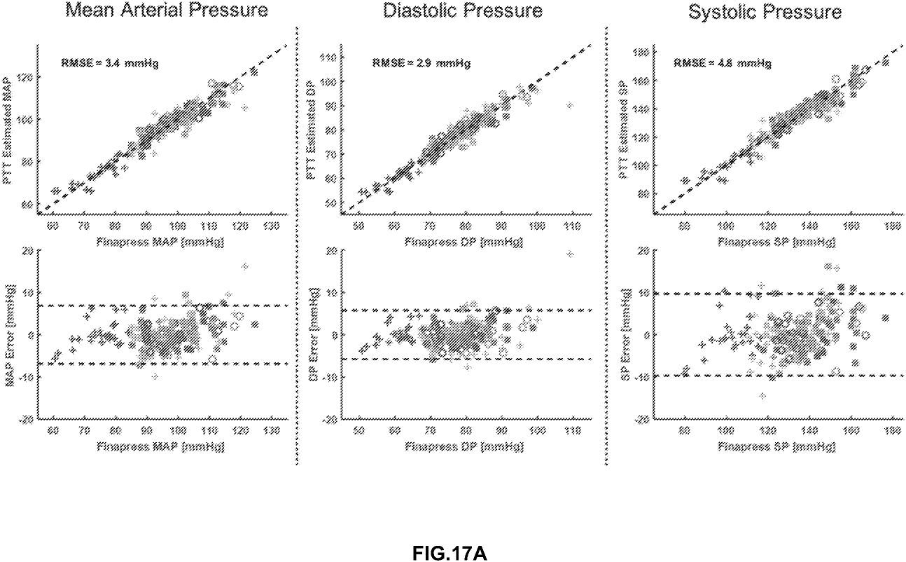

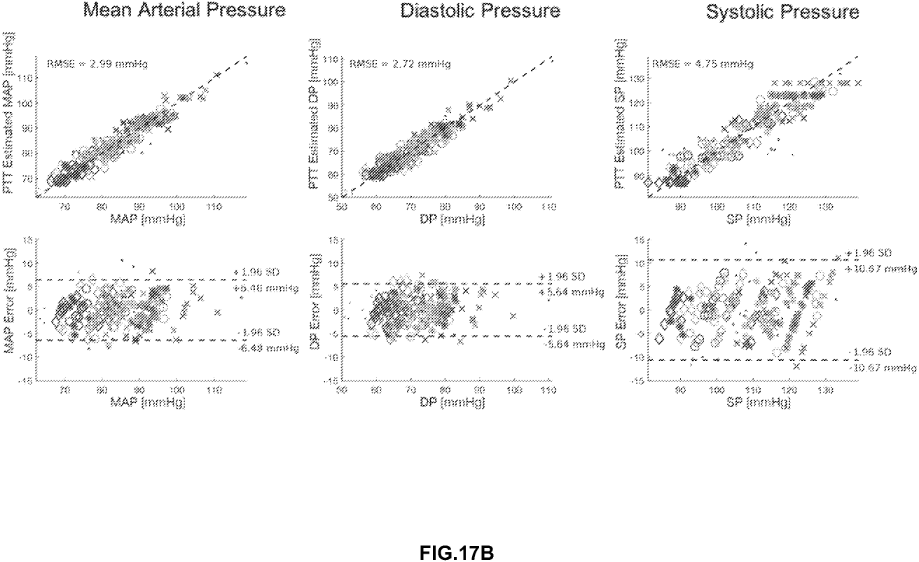

FIG. 17A illustrates the correlation plot and Bland-Altman plots after a best-case calibration of BP based on PTT for MAP, DP, and SP. The RMSE was simply the root mean square of the difference between the PTT estimated BP and the measured BP. The group RMSE for MAP, DP, and SP was 3.2 mmHg, 2.9 mmHg, and 4.8 mmHg, respectively. DP estimations resulted in the best confidence interval (95%) at 5.8 mmHg. SP estimation proved to the least accurate, with the highest RMSE and a confidence interval at 9.7 mmHg. This result is consistent with physiological expectations since the foot of the PPG waveform represents distal pulse arrival which occurs during diastole rather than systole.

Table 1 summaries the individual results of the thirteen subjects when a best-case calibration curve converted PTT to BP. DP formed the lowest error and thus was used for further evaluation. µDP was the average diastolic pressure, RMSE was the root mean square error, R was the correlation coefficient, e < 5 mmHg was the ratio between estimations less than 5 mmHg error to the total number of estimations, and e < 10 mmHg was the same ratio but with 10 mmHg of error. Individual RMSE was less than 5 mmHG for twelve of the thirteen subjects. BP estimation errors were below 5 mmHg in eight subjects. Only one subject of the thirteen had a BP estimation error exceeding 10 mmHg.

TABLE 1

| Subj ect | µDP [mmHg] | RMSE [mmHg] | R | e < 5 mmHg | e < 10 mmHg |
|---|---|---|---|---|---|
| 1 | 80 ± 10 | 3.06 | 0.95 | 0.85 | 1.00 |
| 2 | 81 ± 4 | 2.29 | 0.77 | 1.00 | 1.00 |
| 3 | 76 ± 4 | 2.45 | 0.74 | 0.94 | 1.00 |
| 4 | 73 ± 5 | 2.73 | 0.80 | 0.94 | 1.00 |
| 5 | 80 ± 4 | 1.42 | 0.93 | 1.00 | 1.00 |
| 6 | 61 ± 3 | 1.56 | 0.69 | 1.00 | 1.00 |
| 7 | 60 ± 5 | 2.32 | 0.88 | 1.00 | 1.00 |
| 8 | 76 ± 3 | 1.41 | 0.89 | 1.00 | 1.00 |
| 9 | 85 ± 10 | 6.12 | 0.76 | 0.72 | 0.94 |
| 10 | 78 ± 5 | 1.71 | 0.92 | 1.00 | 1.00 |
| 11 | 73 ± 6 | 1.67 | 0.95 | 1.00 | 1.00 |
| 12 | 77 ± 6 | 3.37 | 0.81 | 0.83 | 1.00 |
| 13 | 88 ± 5 | 2.64 | 0.85 | 1.00 | 1.00 |
| µ | 76 | 2.52 | 0.84 | 0.95 | 1.00 |
| σ | 10 | 1.25 | 0.09 | 0.09 | 0.04 |

### Example 6: Assessing Quantifying Day-to-Day Repeatability in BP Estimation

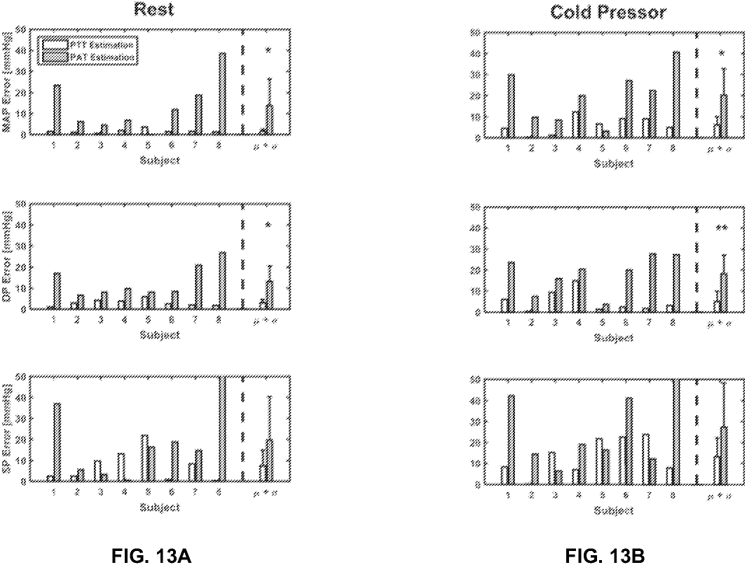

FIGS. 13A and 13B depict the follow-up study testing the day-to-day repeatability of the watch with MAP, DP, and SP estimated with PAT and with PTT. The calibration curves of PTT and PAT to BP were calculated using data from the first protocol and applied the respective curves to PTT and PAT values measured during the follow-up study. During rest, PTT-based BP estimations significantly improved both MAP and DP when compared to PAT-based estimations (p < 0.02 and p < 0.005, respectively). During rest, BP estimations improved by an average of 12.3 mmHg when using PTT-based estimations over PAT-based estimations. All eight subjects had MAP and DP estimations within 5 mmHg of their measured BP levels when using PTT. While the best-case BP using PAT produced less than 1.0 mmHg error, three of the eight estimations produced errors of more than 15 mmHg, rendering the measurements not sufficiently accurate for clinical use. DP had a similar comparison. BP estimations improved by an average of 13.9 mmHg with only one subject greater than 5 mmHg error (e = 5.7 mmHg). In contrast, SP estimations were poor. Though PTT calibration produced errors that were, on average, 12.3 mmHg lower than PAT estimation, no significant difference existed between the two estimations (p > 0.05).

During the cold pressor test, PTT significantly improved both MAP and DP estimations compared to PAT (p < 0.01 and p < 0.005, respectively). However, PTT-based BP estimations worsened when compared to rest values, averaging 6.3 ± 4.1 mmHg error for MAP and 5.0 ± 5.0 mmHg error for DP. Though there was a decline in performance during this perturbation, BP estimation errors were still substantially lower than estimation using PAT which had an average error of 20.1 mmHg for MAP and 18.2 for DP. At the resting phase, the SP estimations were not within an acceptable error. Estimations yielded an average of 13.3 mmHg when using PTT and 27.4 mmHg when using PAT.

### Example 7: Determining Variability in SCG Signal Quality for Unsupervised Settings

Varying the watch in different locations and contact pressures tested the robustness and reliability of the watch in at-home scenarios when trained personnel are not available to supervise. The three contact pressures resulted in similar SCG signals, and the AO peaks only deviated an average of 3.2 ms, well within the normal variability of PEP. Qualitatively, the signal had similar morphology regardless of contact pressure, exhibiting comparable numbers and relative amplitudes of peaks and troughs. However, varying the locations affected the SCG, changing the morphology of the signal based on the location. When the contact area was at the bottom of the sternum, between the sternal angle and the xiphoid process, the signal was consistent and reliable. Positioning the watch above this section distorted the signal, and the AO peak deviated as much as 85%. Furthermore, locations away from the sternum, such as on the softer tissues of the pectoral muscles, resulted in a misclassification of the AO peak. To obtain a reliable SCG signal, the subject will need to place the watch face on the lower portion of the sternum, which should be feasible even in unsupervised settings. Fortunately, methods exist that use classification algorithms to automatically detect when SCG sensors are in any position other than the desired one. Users can be notified of improper placement and to change position. Finally, the presence of light clothing seemed to have little effect on the quality of the signal as the direct skin contact and contact with a light clothing layer produced similar SCG signals.

### Example 8: Reliability of the Watch as a BP Monitor

The systems and methods described herein show promise in being able to conveniently obtain BP measurements outside of clinical settings. A strong correlation between BP and PTT obtained from the timing references derived from an SCG and PPG existed for MAP and DP. While SP did not follow as strong of a trend, this is consistent with previous studies.

When compared to estimations using PAT, the watch showed a significant increase in accuracy. The addition of the accelerometer to measure SCG as the proximal timing reference for PTT proved to be superior to using the ECG as the proximal reference for PAT during rest. While ECG has the advantage of a higher SNR, the confounding effect of PEP negatively impacted MAP and DP estimation. Physical exercise during the calibration phase had a large effect in shortening the PEP. After exercise, cardiac contractility returned to baseline while BP decreased relative to baseline due to exercise-induced vasodilation. Thus, exercise recovery changed BP and PTT proportionally more than PEP. This corrupted the PAT-based calibration curve and led to an underestimation of BP during the follow-up study when BP and cardiac contractility returned to a baseline. In conditions that greatly vary BP but have little effect on PEP, BP estimation with PAT will be poor, and such conditions are common during normal daily living activities.

An increase in error for both PTT and PAT-based estimations were found during the cold pressor when compared to rest, presenting a potential limitation to the current system. The systems described herein measured the pulse wave as it travels through the brachial artery, and the presence of smooth muscle in this arterial path could have accounted for the increase in error between rest and cold pressor. Cold pressor modulated vasomotor tone independent of blood pressure, changing the arterial stiffness and affecting PTT. Though this error occurred, current standards for blood pressure monitoring recommend subjects take measurements at room temperature. Abiding by these recommendations will minimize the impact of the vasomotor tone on PTT.

In the systems and methods described herein, the average power consumption was 360 mW. While this would be difficult to sustain for long periods of time using an average smart watch battery, only a few seconds of measurements were sufficient to obtain a PTT. During the follow-up study, only ten beats were processed, resulting in an average recording time of 8.7 seconds. In future iterations, power consumption was minimized by allowing the user to start the measurement. If there is a need for longer measurements, power consumption could be substantially improved by lowering the output intensity of the LEDs with varying current amplitude or duty cycles. Alternatively, the system could decide on the highest quality LED and turn the other two off.

Compared to a blood pressure cuff, the systems described herein provide a form factor and measurement procedure that is more convenient. During the study, the subjects were given only basic instructions on how to operate the watch. They were instructed on the proper placement of the device before the protocol started and they were successfully able to repeat the maneuver during the entire protocol. BP cuffs, on the other hand, are susceptible to positioning, cuff size, obesity level, and other user errors that would result in inaccurate readings.

Additionally, the subjects could perform the maneuver for five minutes while taking multiple consecutive readings (> 10) without a loss of quality in either the SCG or PPG. The readings were independent of each other, with a measurement having little to no effect on PTT or BP. This would be difficult to achieve with a BP cuff since it is recommended to have one-minute in between successful measurement to prevent the inflation of the cuff, and subsequent occlusions of the artery, from changing the BP at the site of measurement. Thus, dynamic changes in BP in response to stressors such as exercise or mental stress can more readily be quantified with the systems and methods herein.

This work explored the feasibility of using a watch-based wearable device to estimate BP based on PTT. A study was conducted to first establish a strong correlation between watch PTT and BP. A follow-up study tested the consistency of the device and compared the watch PTT method to a PAT method that is commonly used. The results show a significant improvement while using the systems disclosed herein compared to the PAT method. This work established a method of extracting BP that is both convenient and robust enough for at-home usage.

### Example 9: Hardware Design for Home Monitoring

A significant drawback to conventional methods for measuring BP is the lack of portability. To combat this limitation and allow a watch to be used outside the lab, the system and methods of the present disclosure were created. The design was redesigned to incorporate all sensors inside the main body of the watch, removing any need for external components and including an on-board microcontroller, making the watch truly portable. Additionally, to allow for further assessment of cardiac health, an ECG, sternum PPG sensors, gyroscope, and environmental sensor were included. The ECG sensor allows for easy partitioning of the heart beats and can be used to assess the heart rate variability (HRV) to determine autonomic states. To capture sternum PPG, three additional PPG sensors were placed on the top side of the watch to measure the sternum’s pulse wave while the user performs the same maneuver needed to capture the SCG. The sternum PPGs will provide an additional timing reference for PTT calculations. A gyroscope was included to sense the gyrocardiogram (GCG) signal. Error can be reduced when using the GCG in combination with SCG to predict the PEP. The environment sensor measures the temperature, relative humidity, and barometric pressure, adding an aspect of activity context for improved physiological interpretations.

The complete design features three stacked printed circuit boards and a 150 mAh lithium-ion battery inside of a custom 3D printed case. From the backside of the watch― closer to the wrist when worn—to the topside, the boards and battery are stacked in the following order (FIG. 2C): wrist PPG/ECG board, main board, battery, and finally, sternum PPG board. The case includes three slots on both the top and bottom portion to expose the PPG sensors.

For the microcontroller, the ATSAM4LS8B (Microchip Technology, Chandler, AZ) was used for its large amount of storage (512 kBytes Flash, 64 kBytes RAM), high number of peripheral options (48 GPIOs, 4 USART), and ultra-low power consumption (1.5 1aA sleep mode). The custom AFEs used in the previous iteration were replaced with selected sensors with internal AFEs to reduce the number of components and power consumption. Additionally, on-board ADCs were not used due to the relatively high noise and low bit conversion compared to external ADCs. Instead, sensors that included an ADC were used. This allowed for a completely digital interface and allowed the sensors to independently make conversions, freeing up processor time on the microcontroller. Sensors that interface via SPI were also used due to the fast clock speeds (12 MHz).

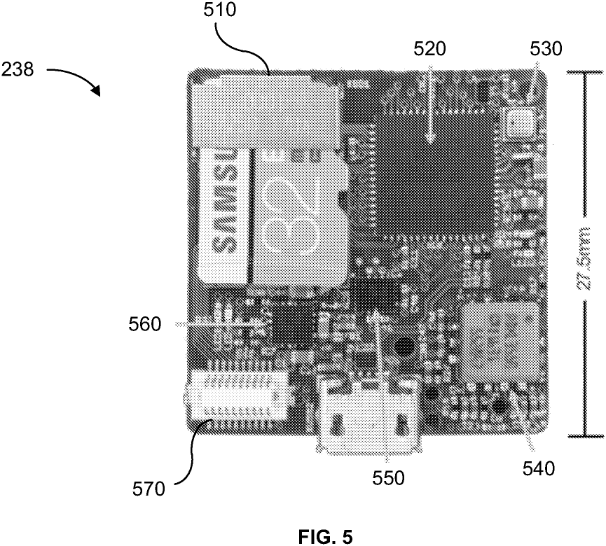

The various components on each of the boards can be seen in FIGS. 3A, 3B, 4A, 4B and 5. The main board (FIG. 5) contains much of the hardware for the watch, including the microcontroller. Additionally, the board includes the charging circuit, accelerometer, gyroscope, environmental sensor, SD card, and various connectors to the other components. For the accelerometer, the digital version of the ADXL354, the ADXL355 (Analog Devices) was selected. The ADXL355 has a noise floor at

$$25 μ g / \sqrt{H z} ,$$

comparable to the

$$20 μ g / \sqrt{H z}$$

of the ADXL355. Additionally, the ADXL355 has a 20-bit ADC with a full-scale range of 3.3 V, a drastic improvement over the MP150′s 16-bit ADC over a 20V range. To measure the GCG, the BMG250 (Bosch) was used due to the low output noise

$$\left(0.007 ° / \text{s} / \sqrt{H z} .\right)$$

The main board also includes the BME280 (Bosch) that features a small package size (2.5 mm x 2.5 mm), low current consumption (3.6 µA), and low noise floor of the pressure sensor (0.2 Pa RMS). The microcontroller stores data on an on-board SD card at a write speed of 12 MB/s.

The wrist PPG/ECG board, as the name implies, contains both the wrist PPG and ECG circuit. On the back side of the board are three SFH7072s (Osram, Munich, Germany) with each containing a green, red, and infrared LED and two photodiodes. One of the photodiodes blocks red and IR wavelength, improving the detection of a green wavelength, while the second photodiode has a peak sensitivity around the red and IR wavelength. Measurements of PTT would utilize the red and infrared detectors to monitor the deeper arteries. The high-SNR green detector could constantly measure heart rate when the user is not taking a PTT measurement and indicate physiological states between PTT measurement. Each SFH7072 interfaces with a MAX86141 (Maxim Integrated, San Jose, CA) to drive the LEDs and to read the current output of the photodiodes. This board also includes the ECG circuitry, including ADS1291 (Texas Instruments, Dallas, TX), selected due to the low-noise (8 µVₚₚ) and high-resolution ADC (24-bit). The ADS1291 connects to three dry stainless-steel electrodes. For the negative reference and the right leg drive, two electrodes are placed on the backside of the watch to make contact with the wrist. A third electrode was placed for the positive reference on the outside of the wristband. The user simply touches the electrode with the opposite hand while taking a measurement. Additionally, the ADS 1291 includes a lead-off detection that constantly monitors the connection to the body. This feature allows the user to initiate a measurement by touching the wristband electrode.

The remaining board, the sternum PPG board, contains three pairs of SFH7060s (Osram) and MAX86140s (Maxim Integrated). The SFH7060 was selected over the SFH7072 due to the increased area of the photodiodes, increasing the total sensitivity and compensating for the decreased perfusion at the sternum when compared to the wrist. Since the SFH7060 only includes a single photodiode, the one-channel MAX86140 was selected.

The ECG is sampled at 1 kHz, the accelerometer and gyroscope are sampled at 500 Hz, the environmental sensor is sampled at 33 Hz, and each PPG sensor is sampled at 333 Hz, and the data is temporarily saved to the SD card. The watch interfaces with the computer through a microUSB port on the main board and is accessible through a cut-out in the case. The HeartPulse App (Department of Anesthesiology, Northwestern Medical, Chicago, IL) communicates with the microcontroller to pull and subsequently delete data on the SD card, freeing up space for future measurements. Additionally, the inserted microUSB interfaces with a battery charger (BQ24232RGTR, Texas Instruments) to charge the battery.

### Example 10: Device Operations and Measurement Modes

The system of the present disclosure was designed to operate in three modes: standby, continuous, and PTT measurement mode. During standby mode, all sensors on the device are shut down except for a PPG on the wrist, and the watch waits for an interrupt from a PPG which was configured to act as a proximity sensor. When an object approaches the sensor, as when the user places the watch on the wrist (FIG. 6B) an interrupt flag is set, and the watch transitions to continuous mode. During this mode, the green PPGs, accelerometer, and gyroscope are active and sample at 125 Hz. The environmental sensor is also turned on and samples at 4 Hz. The lower sample rate saves power while providing enough context for activity classification.

The watch transitions to PTT measurement mode when the ECG senses a lead-on event. When wearing the watch on the wrist, the user will need to touch the wrist-band electrode with hand contralateral to the watch (as seen in FIG. 6A). During this mode, all sensors are sampled at the full rate as previously described. FIG. 9 shows the ensemble average of the ECG, SCG, GCG, and PPG, during a 30-second recording while the watch was operating in the PTT measurement mode.

FIGS. 10A and 10B show the recordings of just SCG and sternum PPG signals (FIG. 10A) and all sensors (FIG. 10B) during a 10-minute walk outdoors. When the user touched the wrist electrode, the watch successfully transitioned to PTT measurement mode, increasing the sample rate and sampling from all sensors. When the finger was removed, the watch returned to continuous mode, decreasing the sample rate and only sensing from sensors that give activity context for determining physiological states. Configuring the watch to transition between these modes reduces power consumption, reduces memory needs, and indicates timings of PTT measurements.

A watch-based system was developed to measure PTT after recording the SCG at the sternum and PPG at the wrist during a simple maneuver. The device was tested over different days and showed an improvement in BP estimation over wrist-based PAT methods. The system was modified to include additional sensors for improved hemodynamic tracking during normal daily living activities. External components were removed such that the device was completely portable with a similar form factor to commercially available smart watches.

A primary limitation of the convention devices for measuring BP is the need for a cuff-based BP measurement for calibration during the first use and periodical updates to account for slow changes in arterial stiffness. A solution could leverage posture-induced changes in hydrostatic pressure to calibrate the systems and methods described herein. The subjects would adjust their arms to a specific height above and below the heart, varying the hydrostatic pressure in the arteries. Both BP and PTT would change, and calibration would be possible. This method requires an ECG and SCG to measure PEP which would be assumed to remain constant during the remaining maneuvers. Then, by measuring the PAT at various levels using the ECG and PPG, it is possible to derive changes in BP and PTT for calibration. A BP cuff reading would still be necessary for the initial calibration, but further measurements would not require a BP cuff reading.

Another drawback of conventional systems is that the user is constrained to perform the maneuver while static. Although the system and method described herein requires direct contact to the sternum by the device due to the SCG’s low signal and poor coupling to the rest of the body, this is substantially improved portability than current cuff-based measurements. Although PTT cannot be constantly monitored, the green wrist PPGs and inertial sensors can continuously record heart rate and activity levels. These sensors would determine the optimal time to take a measurement, such as periods of high heart rate or high activity, and the watch can indicate to the user to take a reading.

Using data from multiple days and various perturbations, supervised learning techniques could estimate the AO based on features of the accelerometer and gyroscope. Furthermore, improvements in the portability enable studies in subjects during normal activities of daily living that are necessary to assess the robustness and the ability to measure PTT in an unsupervised setting.

### Example 11: Natural Variability in BP Over 24-hours

The various components on each of the boards can be seen in FIGS. 2A-2C. Table 2 details the system specifications including the sample rate for each sensor. The main board contains most of the watch hardware: the microcontroller, charging circuit, accelerometer, gyroscope, environmental sensor, SD card, and various connectors to the other components. For the accelerometer, the ADXL355 (Analog Devices, Norwood, MA) that has a noise floor at 25 µg/√Hz and resolution of 0.003 mV/bit was selected. This high-resolution, low-noise accelerometer is needed to accurately measure the SCG, which typically has a peak-to-peak amplitude of 8 mg. To measure the GCG, the BMG250 (Bosch, Gerlingen, Germany) was used due to the low output noise (0.007°/s/√Hz). The main board also includes the BME280 (Bosch, Gerlingen, Germany) that features a small package size (2.5 x 2.5 mm), low current consumption (3.6 µA), and a low noise floor pressure sensor (0.2 Pa RMS). The microcontroller stores data on an on-board SD card at a write speed of 12Mb/s.

TABLE 2

| Parameter | Value | | |
|---|---|---|---|
| | PTT Measurement Mode | Continuous Mode | Standby Mode |
| Data Storage Capacity | (Depends on µSD card size) | | |
| Power Consumption | 19 mA | 5 mA | 4 mA |
| Battery Life (based on 150mAh battery) | 8 hrs | 30 hrs | 38 hrs |

| Physiological Measurements | | | |
|---|---|---|---|
| ECG (single-lead) | | | |
| Bandwidth | 125 Hz | - | - |
| Noise | 8 µVpp | - | - |
| Sample Rate | 1 kHz | - | - |

Accelerometer (3-axis)

| | | | |
|---|---|---|---|
| Bandwidth | 125 Hz | 31.25 Hz | - |
| Noise | $25 μ g / \sqrt{H z}$ | $25 μ g / \sqrt{H z}$ | - |
| Sample Rate | 500 Hz | 125 Hz | - |
| Gyroscope (3-axis) | | | |
| Bandwidth | 125 Hz | 25 Hz | - |
| Noise | $0.007 ° / \text{s} / \sqrt{H z}$ | $0.007 ° / \text{s} / \sqrt{H z}$ | - |
| Sample Rate | 500 Hz | 125 Hz | - |

| Sternum PPG (Green, Red, IR) | | | |
|---|---|---|---|
| Wavelength (λₚₑₐₖ) | 530, 660, 950 nm | -, -, - | -, -, - |
| Spectral Sensitivity | 0.27, 0.47, 0.77 A/W | -, -, - | -, -, - |
| Radiant Sensitive Area | 1.3 x 1.3 mm | -, -, - | -, -, - |
| Sample Rate | 333,333,333 Hz | -, -, | -, -, - |
| Wrist PPG (Green, Red, IR) | | | |
| Wavelength (Aₚₑₐₖ) | 526, 660, 950 nm | 526, -, - nm | 526, -, - nm |
| Spectral Sensitivity | 0.31, 0.56, 0.84 A/W | 0.31, -, - A/W | 0.31, -, - A/W |
| Radiant Sensitive Area | 0.89 x 0.89 mm | 1.29 x 2.69 mm | 1.29 x 2.69 mm |
| Sample Rate | 333, 333, 333 Hz | 125, -, - Hz | 8, -, - Hz |
| Environmental Measurements | | | |
| Temperature Sensor | | | |
| Noise | 0.004° C. RMS | 0.004° C. RMS | - |
| Sample Rate | 33 Hz | 4 Hz | - |
| Pressure Sensor | | | |
| Noise | 1.3 Pa RMS | 1.3 Pa RMS | - |
| Sample Rate | 33 Hz | 4 Hz | - |
| Humidity Sensor | | | |
| Noise | 0.07%RH RMS | 0.07%RH RMS | - |
| Sample Rate | 33 Hz | 4 Hz | - |

*Note that a “-” indicates that particular sensor was not enabled during that measurement mode.

The wrist PPG/ECG board, as the name implies, contains both the wrist PPG and ECG circuit. On the back side of the board are three SFH7072s (Osram, Munich, Germany) with each containing a green, red, and infrared (IR) light-emitting-diode (LED) and two photodiodes (PDs). One of the PDs blocks red and IR wavelengths, improving the detection of a green wavelength, while the second more broadband PD has a peak sensitivity around the red and IR wavelength. Measurements of PTT would utilize the red and IR detectors to monitor the deeper arteries. The high signal-to-noise ratio (SNR) green detector could constantly measure heart rate when the user is not taking a PTT measurement and indicate physiological states between PTT measurements. Each SFH7072 interfaces with a MAX86141 (Maxim Integrated, San Jose, CA) to drive the LEDs and to read the current output of the PDs. This board also includes the ECG circuitry where the ADS1291 (Texas Instruments, Dallas, TX) was selected due to the low-noise (8 µVₚₚ) and high-resolution ADC (24 bit). The ADS1291 connects to three dry stainless steel electrodes. For the negative reference and the right leg drive, two electrodes are placed on the backside of the watch to make contact with the wrist. A third electrode was placed on the outside of the wristband for the positive reference. Using the ADS1291′s lead-off detection feature, which constantly monitors the connection to the body, the user can initiate a measurement by simply touching the wristband electrode with the opposite hand.

The remaining board, the sternum PPG board, contains three pairs of SFH7060s (Osram) and MAX86140s (Maxim Integrated, San Jose, CA). The SFH7060 was selected over the SFH7072 due to the increased area of the PDs, increasing the total sensitivity and compensating for the decreased perfusion at the sternum when compared to the wrist. Since the SFH7060 only includes a single PD, the single-channel MAX86140 AFE was selected. Similar to the SFH7072, the SFH7060s still contain green, red, and IR LEDs.

The data is temporarily saved to the SD card. The watch interfaces with the computer through a microUSB port on the main board and is accessible through a cut-out in the case. A custom C# based app communicates using the USB protocol with the microcontroller to pull and subsequently delete data on the SD card, freeing up space for future measurements. Additionally, the inserted microUSB interfaces with a battery charger (bq24232, Texas Instruments) to charge the battery.

### Example 12: Device Operations

The systems and methods described herein was designed to operate in three modes: standby, continuous, and PTT measurement mode. During standby mode, all sensors on the device are shutdown except for a single green PPG on the wrist which is sampled at 8 Hz and configured to act as a proximity sensor. When an object approaches the sensor, as when the user places the watch on the wrist, an interrupt flag is set, and the watch transitions to continuous mode. During this mode, the three green wrist PPGs, accelerometer, and gyroscope are active and sample at 125 Hz. The environmental sensor is also turned on and samples at 4 Hz. The lower sample rate saves power while providing enough context for activity classification. The watch transitions to PTT measurement mode when the ECG senses a lead-on event. To trigger this mode when wearing the watch on the wrist, the user will need to touch the wrist-band electrode with the hand contralateral to the watch (as seen in FIG. 12). During PTT measurement mode, the sternum PPGs are turned on and the device was placed directly in contact with the subject’s skin at the mid sternum. All wavelengths (i.e., green, red, and IR) of both the wrist and sternum PPGs were activated. The sternum was chosen primarily for optimal SCG quality based on some of previous work characterizing signal quality at different sensor placement locations. In this mode, all sensors are sampled at the full rate as shown in Table 2 above. FIG. 9 shows the ensemble average of the ECG, SCG, GCG, and PPG during a 30-second recording while the watch was operating in the PTT measurement mode.

FIG. 10B shows the recordings of all sensors during a 10-minute walk outdoors. When the user touched the wrist electrode, the watch transitioned to PTT measurement mode, increasing the sample rate and sampling from all sensors. When the finger was removed, the watch returned to continuous mode, decreasing the sample rate and only sensing from sensors that give activity context for determining physiological states. Configuring the watch to transition between these modes reduces power consumption and memory requirements, as well as indicating PTT measurement timings.

To compensate for variable PPG signal quality due to different skin tones amongst subjects, the LED drive current was automatically adjusted for the individual PPGs and wavelengths to prevent railing and improve signal quality. This required having a two-state current threshold and adaptively decreasing the LED current and switching to the lower threshold value if the most significant byte (MSB) of the input light measured from the PD exceeded the higher threshold. Otherwise, the current cutoff was increased to its higher value to allow the signal to grow in amplitude.

### Example 13: Study Protocol

This study was conducted under a protocol approved by the Institutional Review Board (IRB). For this study, 21 (16 males, 5 females) young and healthy volunteers (Age: 25.9±3.4 years, Weight: 74.4±16.9 kg, Height: 176.3±10.9 cm, and BMI: 23.7±3.6) with no prior history of heart problems were recruited, and written informed consent was obtained. The subjects were instructed to take the watch and an Association for the Advancement of Medical Instrumentation (AAMI) approved BP785N BP cuff (Omron, Kyoto, Japan) home and acquire measurements hourly over the course of 24 hours, taking at least 12 total measurements. In addition, they were told to specifically take a measurement directly before and after bedtime and to not wear the watch during sleep. Therefore, the watch was worn until bedtime to obtain data from the continuous mode-for activity context was charging during sleep and re-worn the next morning. The subjects were briefed on how to use the watch and BP cuff to perform measurements. Since all the data was saved automatically from both devices the protocol required low-user-input, essential for an at-home measurement system. Every measurement session consisted of five sections broken down into three 30 second watch measurements with BP cuff measurements in between as shown in FIG. 12, resulting in approximately three-minute-long measurement sessions. At least 15 seconds were added between the middle watch measurement and the BP cuff measurements before and after to comply with the American Heart Association’s (AHA’s) recommendation of one-minute intervals between BP cuff measurements.

### Example 14: Signal Processing

Only the data from the PTT measurement mode were analyzed in this work. The entire recording was partitioned using the serviceable ECG lead-on detection feature to extract signals from each of the individual measurement sessions. The length of signals was reduced to 15 seconds per session for PTT analysis to remove sections corrupted with motion artifacts caused by the subject still adjusting and placing the watch on the sternum. Additionally, the sternum and all green and red wavelength PPGs were not utilized as the IR wavelength wrist PPGs had the highest mean SNR, potentially due to IR wavelength’s ability to penetrate deeper in the tissue and capture larger, more pulsatile arteries. Furthermore, only data from the last cuff reading and measurement session were used because the subject had a greater likelihood of reaching a resting steady-state before readings. Since the subjects did not take both measurements at the same time, using the later measurements when the subject was more likely to have a constant BP allowed us to more accurately compare cuff BP to watch PTT. However, a few subjects had corrupted watch measurements due to erratic toggling probably due to misapplication of the ECG. In those cases, the latest clean watch section was used. For those first-watch section subjects, the first BP cuff measurement was also used. For the other subjects, the first cuff measurement was used because the second had the lowest recorded standard deviation (SD) of DP at 2.2 mmHg.

The PTT was calculated as the difference between the proximal timing reference, aortic valve opening (AO) point of the dorso-ventral SCG (i.e., z-axis acceleration), and the distal timing reference, foot of highest SNR PPG. First, the ECG, SCG, and PPG signals were filtered using a digital FIR bandpass filter with a bandwidths of 2.2 - 30 Hz, 0.8 - 25 Hz, 1 - 8 Hz, respectively. Then, the SCG and PPG waveforms were split into separate beats by using a simple peak detection algorithm for determining R-to-R intervals of the ECG. Next, the SCG and PPG beats were ensemble averaged and the resulting waveforms were used to extract both of the aforementioned timing references. The SNR was calculated using a noise-to-signal ratio (NSR) detection algorithm, the foot of the PPG was computed from the tangental point method, and the AO point was assumed to be the first peak in each ensemble averaged window. SNR thresholds were set to retain only high fidelity signals; if the SNR of the SCG or PPG beats was not greater than the prescribed cutoff, then the respective ensemble averaged waveforms were deemed too noisy for use. If the SCG or all of the PPG waveforms were discarded, then that measurement session was not used for PTT calculation. If the subject had fewer than 9 measurements with valid SNR levels, the SNR thresholds were gradually decreased in an effort to yield more data points. This approach led to subject-specific SNR thresholds but only yielded at least 75% of total measurement sessions per subject for regression.

### Example 15: Statistical Analysis

Multiple linear regression tests were performed to calculate the coefficient estimates necessary to estimate MAP, DP, and SP from inverse PTT values for each subject. Outliers were removed if the corresponding residual was greater than expected in 95% of new observations. The root-mean-square-error (RMSE) was calculated from the root-mean-square (RMS) of the difference between the estimated BP and measured BP. The error bounds for analysis were chosen based on the AAMI’s guidelines for noninvasive cuff-less BP devices.

### Example 16: Calibration Analysis

For the calibration tests only DP values were examined as research has shown that only DP correlates accurately with PTT [5]. Data points of PTT and corresponding DP values for each subject were randomly selected for training an intra-subject linear regression curve used to determine calibration coefficients. These coefficients were then used to estimate DP based on the PTT values from the remaining data points (i.e., testing set) for that subject. As aforementioned, the RMSE was computed between the estimated and measured DP. This process was repeated until the averaged RMSE reached an expected value. An increasing number of training points were used, which were again independently and randomly selected from each subject. Data are shown until a total of six training points as there were subjects with only seven total data points. Since regression was not possible based on a single training point, the first randomly selected data point’s DP value would be the same estimated DP value for all of the subject’s points was assumed for single-point calibration, hence the labeling ‘Constant BP.’

To test the performance of a semi-globalized model to estimate DP from PTT, global constraints were placed on either the slope or y-intercept calibration coefficient while adaptively changing the other. The individual global methods either fix the testing subject’s y-intercept or slope to the y-intercept or slope output of the linear regression curve calculated from all the PTT and DP data points for all the remaining subjects, respectively. The other calibration coefficient was estimated for each subj ect independently using this global constraint for an increasing number of calibration points.

Two two-point calibration methods were tested and compared to the regular multi-point calibration already presented in this work. For these methods either both the PTT values associated with the maximum and minimum BP or the maximum and minimum PTT values over the subject’s 24-hour period were chosen for use in training a linear regression curve for BP estimation. Subjects were removed if BP and PTT values used for calibration directly correlated. This resulted in only one subject being removed, and only for the BP dynamic range method. The other subject that was removed from both methods was identified as an outlier probably due to having the lowest Pearson correlation coefficient (r = 0.44) of the entire population. Two-sample t-tests were performed for these various calibration analyses to assess the significance (p<0.05) level of these differences in RMSE from independent, randomly sampled points with normal distributions.

### Example 17: 24-Hour At-Home Device Results

FIG. 3 illustrates the correlation and Bland-Altman plots for PTT-based BP calibration of MAP, DP, and SP across all subjects. A more thorough view of the results per subject are provided in Table 3, below. The mean±SD RMSE was 2.72±0.75 mmHg, 2.99±1.12 mmHg, and 4.75±2.29 mmHg for DP, MAP, and SP, respectively. DP and MAP estimation had better confidence intervals (95%) than SP at 5.64 mmHg, 6.48 mmHg, and 10.67 mmHg, respectively. The Pearson correlation coefficients were 0.69, 0.61, and 0.33 for PTT-based DP, MAP, and SP estimation respectively. Across all subjects, data from 216 out of 245 total measurement sessions (88%) was used, with at least 75% of measurements used per subject. All unused measurement sessions were deemed too noisy for a trustworthy PTT calculation.

TABLE 3

| Subject | µDP [mmHg] | RMSE [mmHg] | R | e < 5 mmHg |
|---|---|---|---|---|
| 1 | 77 ± 3 | 1.09 | 0.92 | 1.00 |
| 2 | 79 ± 3 | 2.31 | 0.54 | 1.00 |
| 3 | 64 ± 5 | 4.07 | 0.48 | 0.70 |
| 4 | 76 ± 6 | 3.01 | 0.84 | 0.89 |
| 5 | 73 ± 5 | 2.81 | 0.76 | 0.80 |
| 6 | 56 ± 4 | 2.27 | 0.80 | 1.00 |
| 7 | 66 ± 5 | 3.13 | 0.72 | 0.89 |
| 8 | 66 ± 3 | 2.87 | 0.45 | 1.00 |
| 9 | 91 ± 5 | 3.49 | 0.71 | 0.90 |
| 10 | 77 ± 5 | 1.99 | 0.89 | 1.00 |
| 11 | 69 ± 4 | 2.61 | 0.67 | 1.00 |
| 12 | 78 ± 3 | 2.36 | 0.48 | 1.00 |
| 13 | 67 ± 4 | 3.54 | 0.54 | 0.82 |
| 14 | 81 ± 4 | 2.90 | 0.50 | 0.90 |
| 15 | 64 ± 4 | 1.64 | 0.90 | 1.00 |
| 16 | 72 ± 3 | 2.18 | 0.72 | 1.00 |
| 17 | 75 ± 4 | 2.70 | 0.64 | 0.89 |
| 18 | 61 ± 3 | 1.73 | 0.75 | 1.00 |
| 19 | 68 ± 5 | 3.64 | 0.69 | 0.83 |
| 20 | 73 ± 4 | 3.28 | 0.44 | 0.78 |
| 21 | 75 ± 12 | 3.50 | 0.95 | 0.69 |
| µ | 72 | 2.72 | 0.69 | 0.91 |
| σ | 9 | 0.75 | 0.16 | 0.10 |

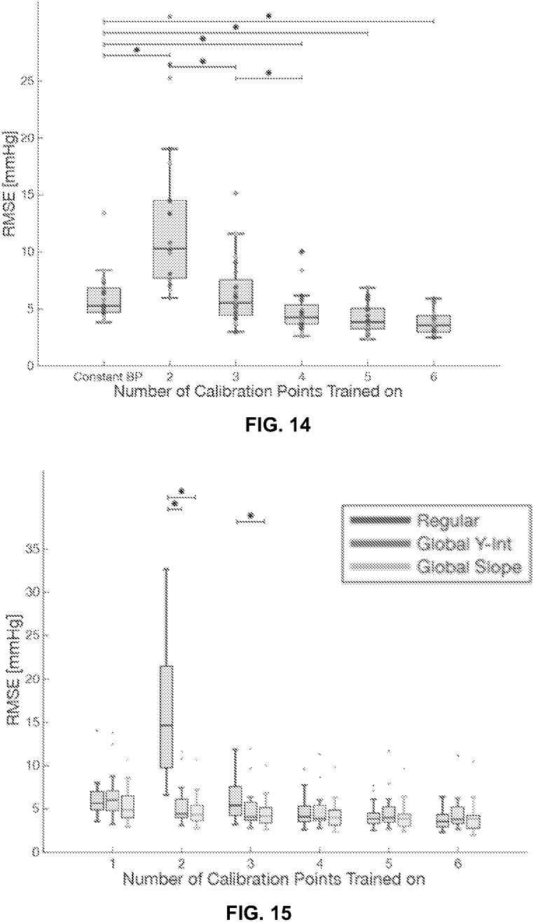

FIG. 14 depicts changes in RMSE across a different number of training points and comparisons between semi-globalized calibration models. Notably, in FIG. 15 RMSE significantly (p<0.05) decreased from a single-point calibration (6.05±1.75 mmHg) to four-point (4.73±2.41 mmHg, p = 0.0495), five-point (4.24±1.97 mmHg, p = 0.0043), and six-point (3.83±1.40 mmHg, p = 0.0004) calibration. Four points were the minimum required for calibration to result in a mean (4.73±2.41 mmHg) that was lower than that of the single-point calibration (6.05±1.75 mmHg). Despite not showing statistical significance, the globalized slope calibration model in FIG. 15 outperformed the regular intrasubject calibration with one point (5.34±1.59 mmHg vs. 6.05±1.75 mmHg). However, at four and six points the regular intrasubject calibration model began to outperform the global y-intercept model (4.73±2.41 mmHg vs. 4.81±1.22 mmHg) and global-slope model (3.83±1.40 mmHg vs. 4.03±1.28 mmHg) respectively.

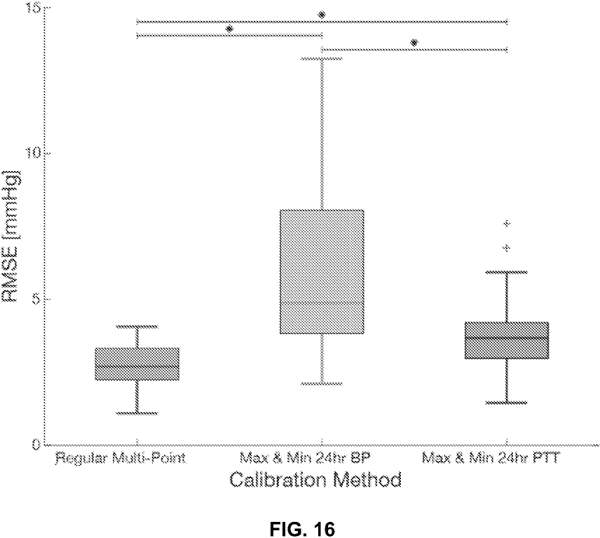

FIG. 16 shows the box-plots of two different two point calibration methods compared to the regular multi-point calibration method. The mean±SD values for the regular multi-point, BP dynamic range, and PTT dynamic range methods are 2.71±0.75 mmHg, 5.99±3.03 mmHg, and 3.86±1.53 mmHg respectively. Both dynamic range two-point calibration methods significantly outperformed the regular, randomly selected, two-point intrasubject testing loss (11.62±16.82 mmHg) seen in FIG. 14.

The effectiveness of PTT-based BP in an uncontrolled setting, where external stimuli can modulate vasomotor tone independently from BP and generate unbalanced peripheral arterial elasticity which relates to PTT. Over the course of a day, several encounters and stressors can instantaneously modulate sympathetic arousal to different degrees acutely affecting vascular smooth muscle tone prior to and independently from BP, transiently confounding PTT but not BP cuff measurements. Additionally, during common daily activities such as consuming caffeine, arterial stiffness increases acutely and independently from changes in BP. Even with these diverse factors—intrinsically coupled to an around-the-clock study-affecting vascular tone independently from BP and confounding PTT measurements, the systems and methods described herein were able to get a reliable correlation between PTT and BP. DP estimation proved to be the most accurate. The foot of the PPG waveform signifies the arrival of the distal pulse wave and signifies end-diastole. Meanwhile, measurements based on SP estimation performed the worst, as the peak rather than the foot of the pulse wave was more related to systole. However, a few studies have shown that detecting systolic peaks is more error prone due to false identification of the reflected pulse wave. It is noteworthy that the subject with the highest Pearson correlation coefficient (subject = 21, r = 0.95) also had the greatest standard deviation of DP (± 12). Therefore, the systems and methods herein prove to be a promising possible indication that it would be best to acquire fewer, more significant, calibration points after some initial exercise or when BP has changed past a significant threshold from baseline to capture the full dynamic range of normal daily BP.

### Example 18: Two-Point Calibration Method

Two-point calibration methods and semi-globalized adaptive calibration models for measuring BP are based on PTT measurements described herein.

This data suggests that even with only four points- on average less than half of the used data points for the multi-point calibration-this device could predict BP over the course of a day within the AAMI’s standards. Due to outliers stemming from the inclusion of noise and lack of BP range, the regression-based approach for two and three-point calibration performed worse than single-point calibration. Furthermore, given the method for single-point calibration, this graph depicts both the need for computing a Pearson correlation coefficient-a metric that no previous PTT-based study measuring natural variability in BP has provided-and the difficulties associated with obtaining a higher correlation with such a small dynamic range in measurements.

To reduce the need for calibration points, global model parameters were incorporated into subject-specific models. These integrated models were better estimators of BP than the regular intrasubject multi-point method with a fewer number of calibration points. Understandably, the multi-point subject-specific calibration method eventually surpassed the global models. Additionally, the global slope model outperformed the global y-intercept model considerably and consistently, as well as the regular intrasubject testing loss until six points, though not significantly other than at three calibration points. Compared to a globalized model, a subject-specific model will realistically always have improved performance, with the downside of increased complexity. However, in the meantime this result shows potential in the feasibility of reducing this complexity through fewer calibration requirements. A heuristic approach that represents an intermediate-globalized model can be adaptively and marginally tailored to a specific subject to provide a more accurate estimation when using fewer calibration points for estimation. In addition, the two-point calibration methods greatly improve upon the intrasubject two-point calibration testing loss seen in FIGS. 14 and 15. This can be attributed to a training set with increased BP range, thereby avoiding randomly selected points that neighbor one another and thus do not offer a good assessment of normal daily BP range. Markedly, the PTT dynamic range method shows statistically significant improvement when compared to the BP dynamic range method, potentially due to accounting for differences in vasomotor tone confounding PTT-based BP estimation. These results become especially important when considering the ability of the hardware to use a multi-modal sensor fusion approach to gate different operational modes and prompt users for more physiologically salient calibration measurements. That is, the fewer the calibration points required, the greater the time the device can spend operating in low-power states providing a more optimal device for the monitoring of hypertension throughout the day remotely. In order to decrease computation even more, this data suggests that a recursive least-squares approach could be applied to a subject’s past data to optimally estimate a calibration curve given a new point. Finally, once enough datasets amongst at-home, longitudinal, and at-risk monitoring subjects have been collected, these similar methods could be reanalyzed and combined with more recent work that has shown promise in estimating blood pressure either through analytical models or machine learning approaches for a potential globalized model.

Compared to an oscillometric cuff, this unobtrusive, watch-based wearable device was conducive to true at-home BP monitoring in an uncontrolled setting and offered a more convenient way to take PTT measurements. The increased convenience is intrinsic to the portable design that does not employ oscillometry which occludes the vessel, causing not only increased measurement error in back-to-back readings, but also discomfort. As a result, the device may increase user compliance, addressing the need for a more widespread adoption of remote BP monitoring. Additionally, the maneuver where the user places the watch on their sternum benefits from being at a heart level which helps increase BP reading accuracy from wrist-worn devices. Though there is a need for continuous BP monitoring in a reliable device that is usable enough to feasibly acquire multiple around-the-clock BP measurements, more conveniently than a BP cuff, providing a clinician a sufficient assessment of normal daily BP variability.

The need for different SNR cutoffs when analyzing data stemmed from low SNR signals which could be attributed to different skin tones—despite auto-optimization of drive currents-placement errors, and an overall higher intersubject and intrasubject variability in an at-home, around-the-clock, unsupervised study. Despite these factors, the device disclosed herein allowed for reliable measurements as all of the measurements were well within the ranges set by the AAMI (µ ≤ 5 mmHg and σ ≤ 8 mmHg). The significance of this work lies in its unobtrusive, reliable, and wearable noninvasive BP estimation. These three integral features of the device demonstrate its promise in addressing the need for an around-the-clock, at-home device capable of consistent normal daily BP monitoring—a major contribution towards diagnosing and managing hypertension remotely.

There does not exist a device currently in research or on the market that combines a multi-wavelength, multi-location PPG array, high-resolution ECG, and ultra-sensitive accelerometer and gyroscope into a compact device that can be used for BP estimation. Furthermore, only because of this unique combination of sensing modalities, a novel firmware was developed that uses multiple sensors to switch between different operational modes that extend battery life—an emerging requirement for wearable devices. The device has a power consumption low enough to easily acquire PTT data for a complete assessment of around-the-clock BP changes. This was enabled through the additional capability of transitioning between device modes (PTT measurement, continuous, standby mode) that allowed the device save battery life (8, 30, and 38 hrs) and reduce memory requirements. If used as an ambulatory BP cuff, which takes measurements every 30 minutes, the watch would only be in measurement mode 0.8% of the time with 15 second measurements. Thus, battery life is minimally affected by power used during PTT measurement mode and could be greatly improved by simply decreasing power consumption during continuous and standby mode through the use of sleep states already built-into the hardware.

One of the challenges of using the SCG as the proximal timing reference is determining the true AO peak. The signal varies greatly between subjects, and the peaks can either be mistaken for the wrong physiological markers or the location of the peaks can be corrupted by either motion artifacts or improper placement of the watch. The method to determine the AO point led to a high correlation between PTT and BP, for a few sessions it annotated the incorrect AO point and had to be manually remedied. At-home monitoring of natural variability in BP is a more challenging problem and dictates a need to capitalize on promising signal processing advances. Specifically, by using new machine learning (ML) approaches to assess signal quality or a gyroscope as an additional proximal timing reference.

To improve the distal timing reference, an array of three PPGs was attached to the top side of the watch. These PPGs now allow for a pulse wave measurement at the sternum as the user performs the same maneuver needed to capture the SCG. The sternum PPGs might be more closely related to central aortic pressure, a more significant indicator of hypertension and cardiovascular mortality, and therefore might be less afflicted by smooth muscle contraction. However, first, an in-depth characterization of locations for high sternum PPG signal quality must be completed and cross-examined with SCG quality to determine an overall optimized sensor placement location that mitigates potential differences in locations that present with high PPG, SCG SNR. Additionally, there are PPG signal features which will not only allow better selection amongst the various PPGs but also elucidate properties of the underlying waveform morphology that are of greater importance in older, hypertensive populations. For example, features such as the augmentation index and rise-time have been shown to capture physiologically salient pulse waves that generate a more accurate, and meaningful estimated BP.

The quantity of calibration points required can be decreased by selecting ones with more quality and physiological significance. During continuous mode the watch samples the accelerometer, gyroscope, environmental sensor, and green wavelength wrist PPGs which can yield clear hemodynamic activity contextualized information. This feature was included to determine a key time of greater likelihood of BP variability (e.g, via heart rate variability) to prompt for a BP calibration measurement. SCG algorithms utilizing ML approaches should be leveraged to assess positioning and placement inaccuracies to forgo poor measurements or immediately tell the user to readjust for optimal SCG quality and resulting PTT calculation. All of this could not be achieved through the use of a single sensor alone. It is contemplated that reliable subject-specific calibration curves can function across several days, thereby eliminating the need for consistent re-calibration. Nevertheless, pulse wave velocity has been shown to be an independent predictor of longitudinal increases in SP lending credibility to the possibility of attaining a calibration-less option. Specifically, the changes in vasomotor tone, captured by PTT, might also imply that perhaps changes in these calibration factors between successive calibration points can be used to determine changes in arterial elasticity. Insight into longitudinal, consistent, remote monitoring using a PTT-based method might indicate that PTT can notice subclinical changes in arterial elasticity and structure prior to exacerbations such as hypertension and renal failure.

The device can store its data to an SD card. It is contemplated that for remote monitoring, wireless technologies (e.g., cellular, LoRa, Blue-tooth, and Wi-Fi) can be incorporated such that data can be automatically uploaded to the cloud for clinicians to view. A crucial benefit that remote BP monitoring offers is being able to assess treatment efficacy of BP medications for patients at-home, which is significantly more difficult and stressful if patients have to visit a doctor regularly, ironically exacerbating their health status. Therefore, PTT-based BP estimation could be handled on-chip or on the cloud prior to viewing by leveraging advances in lightweight firmware or cloud-based algorithms for computation.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A system for non-invasively measuring blood pressure, the system comprising:
a wearable device having a first surface;
a first sensor configured to receive a first signal, wherein the first signal is indicative of a first blood-volume change in a first vessel of a subject;
a second sensor configured to receive a second signal, wherein the second signal is indicative of a cardiac mechanical motion of the subject; and
a processor configured to generate an output based at least on the first signal and the second signal, the output representing a blood pressure measurement of the subject.

2. The system of claim 1 further comprising:
an actuator configured to emit a measuring signal; and
a third sensor positioned on a second surface of the wearable device;
wherein the received first signal based at least in part on the emitted measuring signal.

3. The system of claim 1, wherein the first vessel is selected from the group consisting of a peripheral vessel of an ear, a nasal septum, a forehead, a sternum, a fingertip, a wrist, a toe, a foot, and any combination thereof, of the subject; and
wherein the first sensor comprises a photodetector, and wherein the first signal comprises light.

4-5. (canceled)

6. The system of claim 2, wherein the actuator comprises a light source; and
wherein the first signal and the measuring signal comprise one or more wavelengths of light.

7-9. (canceled)

10. The system of claim 2, wherein the third sensor is configured to receive a third signal;
wherein the third signal is indicative of a second blood-volume change in a second vessel of the subject; and
wherein the received third signal based at least in part on the emitted measuring signal.

11. (canceled)

12. The system of claim 10, wherein the third sensor comprises a photodetectory;
wherein the actuator comprises a light source;and
wherein the third signal and the measuring signal each comprise light.

13-14. (canceled)

15. The system of claim 10, wherein the second vessel is selected from the group consisting of a peripheral vessel of an ear, a nasal septum, a forehead, a sternum, a fingertip, a wrist, a toe, a foot, and any combination thereof, of the subject.

16-17. (canceled)

18. A system comprising:
a first sensor configured to receive a first signal;
a second sensor configured to receive a second signal; and
a processor configured to generate an output based at least the first and the second signals;
wherein at least one of the sensors is contained within, or on a surface of, a wearable watch-based device configured to provide, using the output, noninvasive, cuff-less blood pressure estimation of a subject; and
wherein at least one of the sensors is selected from the group consisting of an accelerometer, a magnetometer, a microphone, a photodetector, a digital camera, an environmental sensor, an electrode, and combinations thereof.

19. The system of claim 18, wherein at least one of the signals is selected from the group consisting of a seismocardiogram (SCG) signal, a gyrocardiogram (GCG) signal, a photoplethysmogram (PPG) signal, an electrocardiogram (ECG) signal, a ballistocardiogram (BCG), an impedance cardiogram (ICG), an impedance plethysmogram (IPG) signal, and combinations thereof.

20. The system of claim 19 further comprising:
a third sensor; and
a fourth sensor;
wherein the first sensor is positioned on a first surface of the wearable watch-based device and configured to the first signal being indicative of a first blood-volume change in a first vessel of the subject;
wherein the second sensor is positioned within the wearable watch-based device and configured to receive the second signal being indicative of a cardiac mechanical motion of the subject;
wherein the third sensor is positioned on a second surface of the wearable watch-based device and is configured to receive a third signal being indicative of a second blood-volume change in a second vessel of the subject;
wherein the fourth signal is indicative of a first electrical activity of the subject;
wherein the processor is further configured to transition the wearable watch-based device from a normal mode of operation to one or more measurement modes of operation;
wherein at least one of the measurement modes of operation is selected from the group consisting of a continuous mode, a pulse transit time (PTT) mode, pulse arrival time (PAT) mode, pre-ejection period (PEP) mode, a blood pressure (BP) mode, and a pulse wave velocity (PWV) mode; and
wherein at least one of:
the transition from the normal mode of operation to the continuous mode comprises a transition of a mode of the first sensor, a mode of the second sensor, and a mode of the third sensor;
the transition from the normal mode of operation to the PAT mode comprises a transition of a mode of the first sensor, a mode of the third sensor, and optionally a mode of the fourth sensor; and
the transition from the normal mode of operation to the PEP mode comprises a transition of a mode of the second sensor and a mode of the third sensor.

21-23. (canceled)

24. The system of claim 20 further comprising a fifth sensor configured to receive a fifth signal indicative of a second electrical activity of the subject.

25. (canceled)

26. The system of claim 20 further comprising a sixth sensor positioned within the wearable watch-based device and configured to receive a sixth signal indicative of a mechanical motion of the subject.

27. (canceled)

28. The system of claim 20, wherein the processor is further configured to correlate the first signal and the second signal to one or more hemodynamic variables.

29. The system of claim 28, wherein at least one of the hemodynamic variables is selected from the group consisting of a pulse transit time (PTT), pulse arrival time (PAT), pre-ejection period (PEP), blood pressure (BP), a pulse wave velocity (PWV), and combinations thereof.

30. The system of claim 28, wherein the processor is further configured to extract a blood pressure reading from one or more of the hemodynamic variables.

31. The system of claim 20, wherein the first surface of the wearable watch-based device is configured to be placed in indirect contact with the first vessel of the subject; and
wherein the second surface of the wearable watch-based device is configured to be placed in indirect contact with the second vessel of the subject.

32. The system of claim 31, wherein the first surface of the wearable watch-based device is configured to be placed in direct contact with a sternum of the subject; and
wherein the second surface of the wearable watch-based device is configured to be placed in direct contact with one or more of the ear, the nasal septum, the forehead, the fingertip, the wrist, the toe, and the foot of the subject.

33-34. (canceled)

35. The system of claim 20, wherein the wearable watch-based device comprises a wristwatch.

36. (canceled)

37. A method for non-invasively measuring blood pressure, the method comprising:
receiving, by a wearable device, a first signal indicative of a first blood-volume change in a first vessel of a subject;
receiving, by the wearable device, a second signal indicative of a first cardiac mechanical motion of the subject;
determining, based on the first and second signals, a blood pressure measurement of the subject; and
outputting the blood pressure measurement of the subject.

38-41. (canceled)

42. The method of claim 37, further comprising receiving, by the wearable device, a third signal, wherein the third signal is indicative of a second blood-volume change in a second vessel of the subject.

43. The method of claim 42, wherein the second vessel comprises a peripheral vessel of an ear, a nasal septum, a forehead, a fingertip, a wrist, a toe, a foot, or any combination thereof, of the subject.

44. The method of claim 43, wherein the first vessel and the second vessel comprise different peripheral vessels of the subject.

45. The method of claim 42, wherein the third signal comprises a second PPG signal of the second vessel.

46. The method of claim 42, wherein the third signal further comprises one or more wavelengths.

47. The method of claim 46, wherein each of the one or more wavelengths of the third signal range from about 1000 nm to about 200 nm.

48-49. (canceled)

50. The method of claim 37, further comprising receiving, by the wearable device, a fourth signal, wherein the fourth signal is indicative of a first electrical activity of the subject.

51. The method of claim 50, wherein the fourth signal comprises an electrocardiogram (ECG) signal of the subject.

52. The method of claim 50, wherein the fourth signal comprises an impedance cardiogram (ICG) signal of the subject.

53. The method of claim 50, wherein the fourth signal comprises an impedance plethysmogram (IPG) signal of the subject.

54. The method of claim 37, further comprising receiving, by the wearable device, a fifth signal, wherein the fifth signal is indicative of a second electrical activity of the subject.

55. The method of claim 54, wherein the fifth signal comprises one of an ECG signal, an ICG signal, or an IPG signal of the subject.

56. The method of claim 37, further comprising receiving, by the wearable device, a sixth signal, wherein the sixth signal is indicative of a mechanical motion of the subject.

57. The method of claim 56, wherein sixth signal comprises a gyrocardiogram (GCG) signal of the subject.

58-69. (canceled)

70. A system for non-invasively measuring blood pressure, the system comprising:
a wearable device having a first surface;
a first sensor positioned on the first surface of the wearable device, the first sensor configured to receive a first photoplethysmograph (PPG) signal;
a second sensor positioned within the wearable device, the second sensor configured to receive a seismocardiograph (SCG) signal;
a third sensor configured to receive one or more of an electrocardiogram (ECG) signal, an impedance cardiogram (ICG) signal, an impedance plethysmogram (IPG) signal, or a gyrocardiogram (GCG) signal; and
a processor configured to determine a blood pressure measurement of the subject based on at least the first PPG signal, the SCG signal, and one or more of the ECG, ICG, IPG, or GCG signals and generate an output representing the blood pressure measurement of the subject.

71. The system of claim 70 further comprising a fourth sensor positioned on a second surface of the wearable device, the fourth sensor configured to receive a second PPG signal;
wherein the second PPG signal is indicative of a blood-volume change in a vessel of the subject different than the first PPG signal;
wherein the processor is further configured to transition the wearable device from a normal mode of operation to one or more measurement modes of operation; and
wherein the one or more measurement modes of operation comprise a continuous mode, a pulse transit time (PTT) mode, pulse arrival time (PAT) mode, pre-ejection period (PEP) mode, a blood pressure (BP) mode, and a pulse wave velocity (PWV) mode.

72-73. (canceled)

74. The system of claim 71, wherein the normal mode of operation comprises detection of the first PPG signal, the SCG signal, and the ECG signal.

75. (canceled)

76. The system of claim 71, wherein the transition from the normal mode of operation to the continuous mode comprises initiating the first sensor, the second sensor, and the third sensor of the wearable device.

77. The system of claim 76, wherein the processor is configured to receive the first PPG signal, the SCG signal, and the ECG signal while in continuous mode.

78. The system of claim 71, wherein the transition from the normal mode of operation to the PTT mode comprises the first sensor, the second sensor, and optionally the fourth sensor of the wearable device.

79. The system of claim 78, wherein the processor is configured to receive the first PPG signal, the SCG signal, and optionally the second PPG signal while in PTT mode.

80. The system of claim 71, wherein the transition from the normal mode of operation to the PAT mode comprises the first sensor, the third sensor, and optionally the fourth sensor of the wearable device.

81. The system of claim 80, wherein the processor is configured to receive the first PPG signal, the ECG signal, and optionally the second PPG signal while in PAT mode.

82. The system of claim 71, wherein the transition from the normal mode of operation to the PEP mode comprises the second sensor and the third sensor of the wearable device.

83. The system of claim 82, wherein the processor is configured to receive the SCG signal and the ECG signal while in PEP mode.

84. The system of claim 71, wherein a blood pressure reading is extractable from one or more of the measurement modes of operation.
